(19) United States
(12) Patent Application Publication
(10) Pub. No.: US 2022/0379022 A1
(43) Pub. Date: Dec. 1, 2022

(54) AGENT DELIVERY SYSTEMS, DEVICES, AND METHODS

(71) Applicant: Thermalin, Inc., Waban, MA (US)

(72) Inventors: Dwight Meglan, Westwood, MA (US); Richard William Berenson, Waban, MA (US); Mervyn Dodson Michael, Indianapolis, IN (US); Charles Chung, Burlingame, CA (US); Daniel Contreras, Burlingame, CA (US); Kimberly Harrison, Burlingame, CA (US); Xue'en Yang, Burlingame, CA (US); Robert Gordon Maurice Selby, Melbourn Royston (GB); Alon Greenenko, Cambridge (GB); J. Christopher Flaherty, Nottingham, NH (US)

(21) Appl. No.: 17/770,747

(22) PCT Filed: Oct. 30, 2020
(86) PCT No.: PCT/US2020/058192

§ 371 Date: Apr. 21, 2022

Related U.S. Application Data
(60) Provisional application No. 62/927,863, filed on Oct. 30, 2019.

(51) Int. Cl. A61M 5/168; A61M 5/172

(52) U.S. Cl. CPC A61M 5/16804; A61M 5/172; A61M 2005/14252

(57) ABSTRACT

A system for delivering an agent to a patient, comprises a delivery device. The delivery device is configured to deliver a first agent to the patient, and comprises a reservoir, a transcutaneous delivery assembly, a pumping mechanism, a control module, a power supply, and a housing. The reservoir stores the first agent. The transcutaneous delivery assembly delivers the first agent to the patient transcutaneously. The pumping mechanism receives the first agent from the reservoir and propels the first agent to the transcutaneous delivery assembly. The control module controls at least the pumping mechanism. The power supply provides energy to at least the control module and the pumping mechanism. The housing surrounds at least the pumping mechanism. Methods of delivering an agent to the patient are also described.

### RELATED APPLICATIONS

This present application claims priority to U.S. Provisional Patent Application No. 62/927,863, entitled “Agent Delivery Systems, Devices and Methods”, filed Oct. 30, 2019, the content of which is incorporated herein by reference in its entirety for all purposes

The present application, while not claiming priority to, may be related to U.S. Pat. No. 9,901,622, entitled “Rapid Action Insulin Formulations and Pharmaceutical Delivery Systems”, issued Feb. 27, 2018, the content of which is incorporated herein by reference in its entirety for all purposes.

The present application, while not claiming priority to, may be related to U.S. patent application Ser. No. 15/869,350, entitled “Rapid Action Insulin Formulations and Pharmaceutical Delivery Systems”, filed Jan. 12, 2018, the content of which is incorporated herein by reference in its entirety for all purposes.

### TECHNICAL FIELD

The embodiments disclosed herein relate generally to systems, devices and methods for delivering one or more drugs or other agents to a patient.

### BACKGROUND

Numerous drugs and other agents are used to treat patients suffering from one or more diseases and/or disorders. For some agents, administration of the agent by a pump or other agent delivery system can provide numerous advantages. There is a need for improved agent delivery systems, devices, and methods.

### BRIEF SUMMARY

According to an aspect of the present inventive concepts, a system for delivering an agent to a patient, comprises: a delivery device configured to deliver a first agent to the patient, the delivery device comprising: a reservoir for storing the first agent; a transcutaneous delivery assembly for delivering the first agent to the patient transcutaneously; a pumping mechanism for receiving the first agent from the reservoir and propelling the first agent to the transcutaneous delivery assembly; a control module for controlling at least the pumping mechanism; a power supply for providing energy to at least the control module and the pumping mechanism; and a housing surrounding at least the pumping mechanism.

In some embodiments, the pumping mechanism comprises a fluidic assembly.

In some embodiments, the reservoir comprises a rigid base and a flexible film cover. The rigid base can comprise a permeation resistant polymer that is compatible with the first agent. The polymer can comprise cyclic olefin copolymer. The flexible film cover can comprise a laminate that is resistant to permeation of one or more substances. The one or more substances can comprise oxygen and/or water. The laminate can comprise cyclic olefin copolymer and/or polychlorotrifluoroethylene. The flexible film cover can comprise a rolling-sock structure. The flexible film cover can comprise at least a portion that resists stretching in at least one direction. The flexible film cover can comprise at least a portion that can stretch.

In some embodiments, the reservoir comprises a fill port. The fill port can comprise a septum and/or a plug.

In some embodiments, the reservoir comprises an access port. The access port can comprise a septum. The reservoir can further comprise a fill port, and the access port can be proximal to the fill port.

In some embodiments, the reservoir comprises a bubble capture element configured to trap bubbles. The reservoir can further comprise an access port, and the bubble capture element can be positioned proximal to the access port.

In some embodiments, the reservoir comprises a transparent window.

In some embodiments, the reservoir comprises the first agent. The first agent can be deposited in the reservoir prior to shipment of the delivery device to the patient. The first agent can be configured to resist degradation. The first agent can be configured to retain at least 95% potency when stored at a temperature at or below 30° C. for a time period of at least 1, 3, 4, 5, 6, 8, and/or 12 months. The first agent can comprise a formulation of an insulin analog. The first agent can be a formulation of an insulin analog and has a concentration of at least 100, 200, 220, 300, and/or 500 Units/mL. The first agent can be a formulation of an insulin analog and has a concentration of at least 220 Units/mL. The first agent can comprise an agent selected from the group consisting of: one or more insulin analogs; glucagon; glucose; dextrose; amylin; ghrelin; estrogen; progesterone; prolactin; testosterone; serotonin; cortisol; triiodothyronine; thyroxine; adrenaline; somatotropin; GCSF; HGF; a GLP-1 receptor agonist; a DPP-4 inhibitors; a meglitinide; an SGLT-2 inhibitors; a sulfonylurea; a thiazolidinediones; an analog of any of these; and combinations thereof. The first agent can comprise an insulin analog formulation, and the reservoir can comprise a size that holds at least 300, 400, 500, 600, and/or 800 Units of the insulin analog formulation. The reservoir can comprise a size that holds at least a 3, 4, 5, 7, and/or 15 day supply of the first agent. The reservoir can comprise a fill volume of less than or equal to 2.4, 2.0, 1.5, 1.3, 1.2, 0.8, 0.2, and/or 0.1 m1.

In some embodiments, the pumping mechanism comprises a mechanism selected from the group consisting of: a piston pump; a syringe pump; a peristaltic pump; a MEMS pump; displacement pump; and combinations thereof.

In some embodiments, the pumping mechanism comprises one or more MEMS components. The one or more MEMS components can comprise a pumping actuator. The pumping actuator can comprise one or more actuator valves. The pumping actuator can comprise one or more pumping drive elements. The one or more pumping drive elements can comprise a piezo actuator. The one or more pumping drive elements can comprise a silicon diaphragm actuator via a micro-machined PZT film. The one or more pumping drive elements can comprise an element with a size of 2.1 mm by 2.2 mm by 0.06 mm. The pumping actuator can comprise between one and five chambers that can be actuated to take in and/or expel contents within the pumping actuator. The pumping actuator can comprise multiple chambers that can be connected by microfluidic channels. The pumping actuator can comprise a chamber with a volume of 5, 10, 15, 20, 30, and/or 50 nL. The pumping actuator can be configured to provide a stroke volume of less than or equal to 50, 25, 15, 10, 5, 2, and/or 1 nL. The pumping actuator can be configured to provide a stroke volume of less than or equal to 30 nL. The pumping actuator can be configured to produce an output pressure of at least 10, 20, 30, 60, 80, 100, and/or 200 kPa. The pumping actuator can comprise one or more agent-contacting surfaces comprising a coating. The coating can comprise a coating type selected from the group consisting of: antimicrobial; antibacterial; antifungal; anti-inflammatory; hydrophilic; hydrophobic; water-resistant; conductive; and combinations thereof. The coating can comprise a thin-film coating. The one or more MEMS components can comprise one or more sensors. At least one sensor of the one or more sensors can comprise a pressure sensor positioned proximate the reservoir. At least one sensor of the one or more sensors can comprise a pressure sensor positioned proximate the transcutaneous delivery assembly. At least one sensor of the one or more sensors comprising a pressure sensor can be configured to operate between a first pressure and a second pressure, and the first pressure can comprise a pressure of less than or equal to 0, −10, −50, −100, −200, −300, and/or −500 kPa, and the second pressure can comprise a pressure of at least 10, 50, 100, 200, 300, and/or 500 kPa. At least one sensor of the one or more sensors can comprise a pressure sensor with a sensitivity of at least −1, 1, 2, 4, 8, and/or 10 kPa. At least one sensor of the one or more sensors can comprise a fluid flow sensor. The fluid flow sensor can comprise a sensor selected from the group consisting of: a differential pressure sensor; a positive displacement flow sensor; a flow velocity sensor; a mass flow sensor; an open-channel flow meter; and combinations thereof. The fluid flow sensor can comprise a hot wire anemometer. The hot wire anemometer can comprise platinum. The hot wire anemometer can comprise one heated wire and two unheated wires. The one or more MEMS components can comprise one or more fluid control valves. The one or more fluid control valves can comprise at least one passive valve configured to prevent backflow. The one or more fluid control valves can comprise at least one passive valve configured to prevent excess forward flow. The one or more fluid control valves can comprise at least two valves arranged to provide redundant safety. The one or more fluid control valves can comprise at least one valve configured to block undesired flow and to resist a pressure of up to 1000, 2000, 5000, and/or 10,000 kPa. The one or more MEMS components can be manufactured using semiconductor fabrication techniques.

In some embodiments, the pumping mechanism further comprises an electronic control module. The electronic control module can comprise a control algorithm configured to pump fluid, sense a fluid delivery parameter, and control flow. The electronic control module can be configured to enter an alert state if pressure, temperature, and/or other system parameter exceeds a threshold. The electronic control module can be configured to stop flow of the first agent if the threshold is exceeded.

In some embodiments, the pumping mechanism comprises a microfluidic routing layer. The microfluidic routing layer can comprise one or more microfluidic channels. The microfluidic routing layer can comprise one or more fiducial marks. The microfluidic routing layer can comprise a needle-seating feature. The microfluidic routing layer can comprise at last one channel with a fileted corner. The microfluidic routing layer can comprise CoC or PMA materials. The microfluidic routing layer can comprise at least a portion with a hydrophilic coating. The delivery device can comprise one or more MEMS components, and the microfluidic routing layer can be bonded to the one or more MEMS components.

In some embodiments, the pumping mechanism comprises one or more flow ports. The one or more flow ports can each comprise a needle. At least one needle can comprise a 34, 32, 30, 28, 25, and/or 24 gauge needle. At least one needle can comprise a steel needle. The pumping assembly can comprise a fluidics assembly, and at least one needle extends at least 0.5, 1, 2, and/or 3 mm from the pumping mechanism. At least one needle can comprise a non-coring needle and/or a needle that atraumatically engages a septum. The delivery device can comprise a microfluidic routing layer, and at least one needle can be bonded to the microfluidic routing layer. The one or more flow ports can comprise a first flow port that can be positioned proximal to the reservoir. The one or more flow ports can comprise a first flow port that can be positioned proximal to the transcutaneous delivery assembly. The one or more flow ports can comprise a first flow port and a second flow port, and the first flow port and the second flow port can be positioned on opposite ends of the pumping mechanism.

In some embodiments, the pumping mechanism comprises a bubble capture element. The bubble capture element can be positioned proximal to the reservoir.

In some embodiments, the pumping mechanism comprises a system in a package. The system in a package can comprise MEMS components that are bonded to a circuit board. The system in a package can comprise control electronics that are bonded to a circuit board. The system in a package can comprise a microfluidic routing layer that is bonded to a circuit board. The system in a package can comprise an assembly that is sealed using semiconductor packaging techniques.

In some embodiments, the pumping mechanism comprises at least a portion with a curved geometry.

In some embodiments, the pumping mechanism is positioned proximate a periphery of the housing.

In some embodiments, the pumping mechanism is configured to produce an output pressure of at least 10, 30, 60, 100, 200, and/or 500 kPa.

In some embodiments, the pumping mechanism is configured to deliver, within one second, a bolus volume of the first agent to subcutaneous tissue of at least 25, 50, 100, and/or 200 nL.

In some embodiments, the transcutaneous delivery assembly comprises a proximal portion including a septum and a distal portion comprises a skin-penetrating element. The distal portion can comprise a tip that can be configured to be placed into a location selected from the group consisting of: subcutaneous tissue; an artery; a vein; a subcutaneously implanted access port; and combinations of these. The tip can be configured to be positioned into a subcutaneously implanted access port with a catheter placed to deliver the first agent into: an artery; a vein; tissue; organ tissue; interstitial space between organs or tissue planes; intraperitoneal space; tissue planes around the peritoneum; and combinations thereof. The distal portion can comprise a cannula. The cannula can comprise a polymeric material. The cannula can comprise an internal metal coil. The cannula can comprise multiple outlets. The cannula can comprise an internal needle.

In some embodiments, the transcutaneous delivery assembly is configured to be inserted into and/or connected to the delivery device at the same time as being inserted into the patient. The system can further comprise an insertion device configured to insert the transcutaneous delivery assembly into the patient, and the insertion device can be further configured to perform the insertion and/or connection of the transcutaneous delivery assembly to the delivery device.

In some embodiments, the control module comprises a microprocessor.

In some embodiments, the control module comprises accessory electronics. The accessory electronics can comprise an electronics module configured to: provide communication between components of the system; store data; regulate power; and combinations thereof. The accessory electronics can comprise an electronics module comprising one or more of: a real time clock; actuator control circuits; and/or sensor control circuits.

In some embodiments, the control module comprises an algorithm configured to control the delivery of at least the first agent. The algorithm can be configured to alter the flow rate of the delivery of the first agent based on fluid flow information and/or fluid pressure information. The algorithm can be configured to determine the flow rate of the delivery of the first agent based on one or more of: current and/or historical data from sensors; previous delivery information; the patient's previous response to agent delivery; a patient disclosed plan such as a plan related to meal size, meal composition, exercise, work activities, stress-inducing activities and/or other activities of the patient; parameters communicated to the control module by a communication device, such as parameters that alter how the algorithm responds to other inputs and/or recognizes patterns in other data; and combinations thereof. The algorithm can comprise a learning algorithm. The algorithm can comprise a learning algorithm selected from the group consisting of: a machine learning algorithm; a parameter-based algorithm where the parameters can be adjusted based on the patient's historical response to receiving the first agent; and combinations thereof. The algorithm can comprise a bias, such as a bias toward under delivery of the first agent. The algorithm can be configured to cause the delivery device to enter an alert state when a system parameter exceeds a threshold. The algorithm can be configured to determine one or more of: type of data stored; method of data storage; location of data storage; and/or method of communication of data to another device.

In some embodiments, the power supply comprises a power supply selected from the group consisting of: battery; capacitor; super capacitor; rechargeable power supply; wirelessly rechargeable power supply; and combinations thereof.

In some embodiments, the power supply comprises a flat geometry. The power supply can be positioned proximate the reservoir and/or roughly parallel to a main plane of the reservoir.

In some embodiments, the power supply comprises an energy supply of at least 50, 100, 500, 1000, and/or 2000 joules of energy.

In some embodiments, the power supply is configured to supply energy to the delivery device to last at least 3, 5, 7, 8, 9 and/or 10 days.

In some embodiments, the housing comprises one or more openings. The one or more openings can be configured to perform a function selected from the group consisting of: drainage; pressure compensation, such as pressure compensation for the reservoir; filling; attachment to and/or actuation by an insertion device; allow insertion of a cannula, such as by an insertion device; allow insertion of an insertable glucose sensor, such as by an insertion device; and combinations thereof.

In some embodiments, the housing comprises a transparent window. The transparent window can be positioned in alignment with the reservoir.

In some embodiments, the delivery device comprises a mass less than 20, 15, 12, 10, 9, and/or 8 grams.

In some embodiments, the delivery device comprises a major axis less than 55, 45, 40, 38, 36, and/or 33 mm.

In some embodiments, the delivery device comprises a height less than 10, 8, 7.5, and/or 6.5 mm.

In some embodiments, the delivery device is configured to deliver the first agent for at least 3, 5 and/or 7 days, such as when the first agent comprises insulin.

In some embodiments, the delivery device is configured to deliver the first agent after being filled and then stored for at least 90, 120, 180, 270, and/or 365 days at a temperature of no more than 30° C.

In some embodiments, the delivery device further comprises at least a first portion that can be rotated. The first portion can comprise the reservoir. Rotation of the first portion can fluidly connect the reservoir to the pumping mechanism. The fluidic connection can occur via an intake port of the pumping mechanism fluidly connecting with an access port of the reservoir. The fluidic connection can occur via an access needle of the pumping mechanism piercing an access septum of the reservoir. The rotation of the first portion can turn on the delivery device and/or initiates a priming of the pumping mechanism. The rotation of the first portion can be activated or otherwise caused by an insertion device. The delivery device can further comprise at least a second portion that can be rotated. The second portion can comprise the reservoir and the pumping mechanism. Rotation of the second portion can fluidly connect the pumping mechanism to the transcutaneous delivery assembly. The fluidic connection can occur by an output port of the pumping mechanism fluidly connecting to a proximal portion of the transcutaneous delivery assembly. The fluidic connection can occur by an output needle of the pumping mechanism piercing a septum of the transcutaneous delivery assembly. The rotation of the second portion can enable priming of the transcutaneous delivery assembly. The rotation of the second portion can be activated or otherwise caused by an insertion device.

In some embodiments, the delivery device further comprises a pad configured to collect froth. The pad can be positioned proximate the transcutaneous delivery assembly and/or an exit port of the pumping mechanism.

In some embodiments, the delivery device and/or another component of the system comprises one or more sensors. The one or more sensors can comprise one or more environmental sensors, such as one or more sensors configured to measure temperature, pressure, and/or humidity. The one or more sensors can comprise one or more sensors configured to measure a parameter of the first agent, such as a bubble sensor, particle sensor, and/or light scattering sensor. The one or more sensors can comprise one or more sensors configured to measure a patient parameter. The patient parameter can comprise a parameter selected from the group consisting of: blood glucose; motion; activity; heart rate; EKG; respiration; EEG; a tissue gas parameter; hormone level; pH; blood oxygen saturation; photospectrographic tissue state; electrodermal activity; and combinations thereof. The sensor can be configured to be positioned on and/or within the dermis, epidermis, and/or subcutis. The sensor can comprise an insertable glucose sensing device comprising a skin-penetrating distal portion.

In some embodiments, the delivery device further comprises a transceiver for sending and/or receiving data to and/or from another device of the system. The transceiver can comprise an element configured to deliver Bluetooth 4.1, 4.2, or greater. The transceiver can be configured to deliver data intermittently.

In some embodiments, the delivery device comprises a skin attachment element. The skin attachment element can be configured to attach the delivery device to the patient's skin for at least 3, 5, 7, and/or 10 days.

In some embodiments, the delivery device and/or the system further comprises a feedback module configured to provide alert and/or other feedback to the patient. The feedback module can be configured to provide feedback in a form selected from the group consisting of: haptic; auditory; visual; light; and combinations thereof.

In some embodiments, the delivery device further comprises a fluid resistance element configured to reduce fluid ingress into the delivery device. The fluid resistance element can comprise a polymer coating and/or other coating.

In some embodiments, the reservoir comprises a first reservoir, and the delivery device further comprises a second reservoir. The second reservoir can be configured to store a second agent. The pumping mechanism can comprise a first pumping mechanism, and the delivery device can further comprise a second pumping mechanism configured to deliver the second agent to the patient. The transcutaneous delivery assembly can comprise a first transcutaneous delivery assembly, and the delivery device can further comprise a second transcutaneous delivery assembly for delivering the second agent to the patient.

In some embodiments, the system further comprises an insertion device. The insertion device can be configured to insert the transcutaneous delivery assembly and/or a sensor through the skin of the patient. The insertion device can be removably attached to the delivery device, to the transcutaneous delivery assembly, or to an implantable sensor. The insertion assembly can comprise one or more needles, such as one or more metal needles. At least one needle can be positioned inside a cannula of the transcutaneous needle assembly prior to insertion of the cannula through the patient's skin. The insertion device can comprise an insertion assembly. The insertion assembly can be configured to perform a function selected from the group consisting of: advance a tip portion of a cannula into the patient; insert a cannula into the patient at an adjustable distance; withdraw an insertion needle from a cannula that has been inserted into the patient; automatically withdraw a needle from the patient consequent to activation of the insertion device; insert a sensor such as a glucose sensor; insert a subcutaneous sensor and/or an intradermal sensor; insert a cannula and a sensor simultaneously; and combinations thereof. The insertion device can be configured to cause the delivery device to electrically connect to an implantable sensor. The insertion device can be configured to create a seal between an implantable sensor and the housing of the delivery device.

In some embodiments, the system further comprises a continuous glucose monitoring device. The continuous glucose monitoring device can be configured to periodically transmit data to the delivery device and/or another component of the system

In some embodiments, the system further comprises a communication device configured to transmit and/or receive data from the delivery device. The communication device can comprise a device selected from the group consisting of: smart phone; cell phone; tablet; laptop computer; computer; personal data assistant; smart watch; special purpose communication device; and combinations thereof. The communication device can be configured to provide information to the patient, such as alert or other information provided visually, audibly, and/or tactilely. The communication device can be configured to receive information from the patient. The information received from the patient can comprise a patient-plan. The communication device can be configured to intermittently communicate with the delivery device. The system can further comprise a glucose monitoring device, and the communication device can be further configured to intermittently communicate with a glucose monitoring device. The communication device can be configured to receive data from the delivery device. The communication device can be configured to receive data in a form selected from the group consisting of: sensor data; first agent delivery data; state information data; and combinations thereof. The communication device can be configured to transmit data to a computer network. The communication device can be configured to transmit sensor data and/or first agent delivery data to the computer network. The communication device can be configured to receive data from a computer network. The communication device can be configured to receive one or more performance parameters for an algorithm and/or data representing a message for the patient. The communication device can be configured to transmit data to the delivery device. The communication device can be configured to transmit data related to a performance parameter of an algorithm and/or a patient-plan. The data the communication device transmits can comprise data previously received by a communication device from a previously used delivery device.

In some embodiments, the system further comprises a computer network. The computer network can be configured to receive data from: a communication device and/or the delivery device. The computer network can be configured to transmit data to: a communication device and/or the delivery device. The computer network can be configured to store data received from another component of the system. The computer network can be configured to transmit data to a caregiver of the patient. The transmitted data can comprise telemetry data. The transmitted data can be provided to: a friend of the patient; a family member of the patient; the patient's clinician; and/or a diabetes educator. The computer network can be configured to transmit data and/or an analysis of data to a payor.

In some embodiments, the system further comprises a fill device configured to deliver the first agent into the reservoir. The fill device can be configured to fill the reservoir at a flow rate of at least 1, 5, 10, 20, and/or 100 mL/min. The fill device can be configured to evacuate gas from the reservoir. The fill device can be configured to insert a plug into a fill port of the reservoir. The fill device can be configured to fill the reservoir while the reservoir is positioned inside the housing. The fill device can be configured to fill the reservoir while the reservoir is positioned inside and/or otherwise proximate an insertion device.

In some embodiments, the delivery device comprises a first delivery device, and the system further comprises at least a second delivery device configured to deliver one or more agents to the patient. The at least a second delivery device can be configured to receive instructions and/or other data from the first delivery device. The second delivery device can be configured to be controlled by the first delivery device. The second delivery device can be configured to transmit data to the first delivery device. The second delivery device can operate independently from the first delivery device. The second delivery device can be configured to deliver the first agent. The second delivery device can be configured to deliver a second agent, and the second agent can be different than the first agent.

According to another aspect of the present inventive concepts, a method of delivering an agent to a patient, comprises: selecting a system according to any one or more claims herein; and delivering the first agent to the patient using the delivery device.

According to an aspect of the present inventive concepts, a system for delivering an agent to a patient comprises: a delivery device configured to deliver a first agent to the patient, the delivery device comprising a reservoir for storing the first agent; a transcutaneous delivery assembly for delivering the first agent to the patient transcutaneously; a pumping mechanism for receiving the first agent from the reservoir and propelling the first agent to the transcutaneous delivery assembly; a control module for controlling at least the pumping mechanism; a power supply for providing energy to at least the control module and the pumping mechanism; and a housing surrounding at least the pumping mechanism.

In some embodiments, the system comprises a learning architecture.

In some embodiments, the delivery device is configured to deliver the first agent to the patient for at least three days. The delivery device can be configured to deliver the first agent to the patient for at least 7 days.

In some embodiments, the delivery device comprises a weight of no more than 20 grams, 15 grams, and/or 10 grams when full with the first agent. The delivery device can comprise a weight of no more than 8 grams when full with the first agent.

In some embodiments, the delivery device is used by the patient at least 90 days, 120 days, 150 days, 180 days, 270 days and/or 365 days after being filled with the first agent. The first agent can comprise insulin and/or an analog of insulin.

In some embodiments, the delivery device is configured to communicate with a second device. The delivery device can be configured to communicate using a secure wireless link, such as a wireless link using Bluetooth (e.g. Bluetooth 4.1, higher and/or equivalent), to BATDOC, Medhub, and/or ACCS. The second device can comprise a glucose sensing device. The glucose sensing device can comprise a continuous glucose sensing device.

In some embodiments, the delivery device comprises a second reservoir configured to store a second agent, and the delivery device is configured to deliver the second agent to the patient. The first agent can comprise insulin and/or an analog of insulin and the second agent can comprise glucagon and/or glucose.

In some embodiments, the delivery device comprises a first delivery device and the system further comprises a second delivery device configured to deliver a second agent. The first agent can comprise insulin and/or an analog of insulin and the second agent can comprise glucagon and/or glucose. The first delivery device and the second delivery device can be configured in a client-server arrangement. The system can further comprise an algorithm configured to control delivery of the first agent by the first delivery device and delivery of the second agent by the second delivery device.

In some embodiments, the delivery device comprises a fluidic connection layer.

In some embodiments, the delivery device includes a first portion configured to be rotated, directly or indirectly, by a user (e.g. by hand and/or using a separate device), in a first rotation step. The first portion can include the reservoir. Completion of the first rotation step can create a fluid pathway between the reservoir and the pumping mechanism. After completion of the first rotation step, the delivery device can be configured to enter an on state and/or perform a priming procedure configured to prime the pumping mechanism. After completion of the first rotation step, the delivery device can be configured to both enter the on state and perform the priming procedure. The delivery device can comprise a second portion configured to be rotated, directly or indirectly by a user (e.g. by hand and/or using a separate device) in a second rotation step. The second portion can include the reservoir and the pumping mechanism. The completion of the second rotation step can create a fluid pathway between the pumping mechanism and the transcutaneous delivery assembly. After completion of the second rotation step, the delivery device can be configured to perform a priming procedure configured to prime the transcutaneous delivery assembly.

In some embodiments, the delivery device comprises a pad constructed and arranged to collect froth produced by the delivery device (e.g. froth produced initially by the delivery device during the pump priming process). The reservoir can comprise a drain port, and the pad can be positioned proximate the drain port.

In some embodiments, the delivery device comprises a major axis less than or equal to 45 mm, 40 mm, 35 mm, or 33 mm.

In some embodiments, the delivery device comprises a height less than or equal to 10 mm, 8 mm, 7.5 mm, or 6.5 mm.

In some embodiments, the delivery device comprises a skin attachment element configured to removably attach the delivery device to the patient's skin.

In some embodiments, the housing comprises a rigid portion and a flexible portion.

In some embodiments, the housing includes a transparent window portion and the reservoir includes a transparent window portion, and the housing transparent window portion is positioned above the reservoir transparent window portion.

In some embodiments, the housing includes one or more openings constructed and arranged to drain fluid from within the housing.

In some embodiments, the reservoir includes a fill port configured to allow the reservoir to be filled with the first agent. The fill port can be configured to allow refilling of the reservoir.

In some embodiments, the reservoir comprises a first reservoir for storing the first agent and a second reservoir for storing a second agent. The first agent can comprise insulin and/or an analog of insulin and the second agent can comprise glucagon and/or glucose.

In some embodiments, the reservoir comprises a rigid base (e.g. a rigid COC base) and a two-layer film cover. The two-layer film cover can comprise PCTFE (e.g. a two-layer film comprising PCTFE and COC) and can be laminated to the COC base to be oriented toward or away from the first agent within the reservoir. In some embodiments, a COC layer is positioned to face the first agent within the reservoir.

In some embodiments, the reservoir comprises a rolling sock design.

In some embodiments, the reservoir comprises two flexible films bonded together.

In some embodiments, the reservoir comprises a flexible film. The flexible film can be constructed and arranged to resist stretching in at least one direction. The flexible film can comprise at least a portion that can be constructed and arranged to stretch.

In some embodiments, the reservoir comprises a full volume of no more than 0.1 mL, 0.2 mL, 0.5 mL, 0.8 mL, 1.0 mL, 1.2 mL, 1.5 mL, or 2.4 mL.

In some embodiments, the system further comprises an algorithm. The algorithm can be configured to compare a measured pressure level of the first agent pressure to an expected level of that pressure during the delivery of the first agent to the patient. The delivery device can enter a warning state if the difference between the measured pressure level and the expected pressure level exceeds a threshold. The algorithm can be configured to adjust and/or otherwise control the delivery of the first agent. The algorithm can comprise a learning algorithm. The algorithm can comprise a machine learning algorithm. The system can further comprise at least one sensor configured to perform a measurement, and the algorithm can be configured to adjust and/or otherwise control the delivery of the first agent based on the measurement. The at least one sensor can comprise one or more temperature sensors and/or one or more pressure sensors. The measurement can comprise a measurement of a patient parameter selected from the group consisting of: glucose level or other patient physiologic parameter; patient motion and/or other patient activity; patient heart rate; patient galvanic skin resistance (GSR) and/or electrodermal activity (EDA); and combinations thereof. The algorithm can be configured to adjust and/or otherwise control the delivery of the first agent based on a patient-disclosed plan. The algorithm can adjust and/or otherwise control the delivery of the first agent based on data from multiple sources. The algorithm can comprise a bias toward under-delivery. The algorithm can comprise a bias toward over-delivery. The algorithm can be configured to cause the system to enter an alarm state when a system and/or patient parameter exceeds a threshold.

In some embodiments, the power supply comprises a battery.

In some embodiments, the power supply comprises a capacitor, such as a supercapacitor. The power supply can further comprise a battery.

In some embodiments, the power supply comprises a rechargeable component. The rechargeable component can be configured to be wirelessly recharged.

In some embodiments, the power supply comprises a flat geometry and is positioned under the reservoir. In some embodiments, the power supply comprises a flat geometry and is positioned on top of the reservoir.

In some embodiments, the power supply comprises a supply configured to provide at least 50, 100, 500, 1000, and/or 2000 joules of energy.

In some embodiments, the power supply is configured to support operation of the delivery device for at least 3 days, 3.3 days, or at least 5.3 days.

In some embodiments, the power supply is configured to support operation of the delivery device for at least 7 days, 7.3 days, or at least 11.3 days.

In some embodiments, the pumping mechanism comprises a pump selected from the group consisting of: MEMS pump; syringe pump; displacement pump; peristaltic pump; and combinations thereof.

In some embodiments, the pumping mechanism is configured to deliver discrete boluses of the first agent. The discrete boluses can comprise a volume of no more than 1 nL, 2 nL, 5 nL, 10 nL, 15 nL, 25 nL, or 50 nL.

In some embodiments, the pumping mechanism comprises surfaces that contact the first agent, and the surfaces comprise a coating. The coating can comprise a coating selected from the group consisting of: antimicrobial; antibacterial; antifungal; anti-inflammatory; hydrophilic; hydrophobic; water-resistant; conductive; and combinations thereof. The coating can comprise a thin film.

In some embodiments, the pumping mechanism is positioned proximate a periphery of the housing.

In some embodiments, the pumping mechanism comprises a MEMS module. The MEMS module can comprise a composite of one or more silicon sensors and actuators assembled on a fluidic substrate. The MEMS module can comprise a curved geometry. The MEMS module can comprise a microfluidic routing layer. The microfluidic routing layer can comprise microfluidic channels. The microfluidic routing layer can comprise one or more fiducial marks. The microfluidic routing layer can comprise a needle-seating feature. The microfluidic routing layer can comprise a channel with a fileted corner (e.g. one, two, or more fileted corners). The microfluidic routing layer can comprise COC and/or polymethyl methacrylate (PMMA) materials. The microfluidic routing layer can comprise a hydrophilic coating. The reservoir can comprise an access septum, and the delivery device can comprise a needle or other penetrating element for penetrating the access septum to provide a fluid pathway between the reservoir and the pumping mechanism. The needle or other penetrating element can comprise a needle (e.g. a 28 gauge needle) that extends at least 1 mm from the MEMS module. The reservoir can comprise a needle or other penetrating element, and the MEMS module can comprise an access septum for receiving the needle or other penetrating element to provide a fluid pathway between the reservoir and the pumping mechanism. The needle or other penetrating element can comprise a needle (e.g. a 28 gauge needle) that extends at least 1 mm from the reservoir. The delivery device can comprise a pressure sensor positioned to measure pressure of fluid proximate the reservoir. The delivery device can comprise a pressure sensor positioned to measure pressure of fluid proximate the transcutaneous delivery assembly. The MEMS module can comprise a MEMS pumping actuator. The MEMS module can comprise a flow sensor. The MEMS module can comprise a passive check valve constructed and arranged to prevent runaway forward flow. The MEMS module can comprise a passive check valve constructed and arranged to prevent backflow. The MEMS module can comprise pumping actuator valves, the MEMS module further comprising an active check valve constructed and arranged to provide a redundancy to the pumping actuator valves. The transcutaneous delivery assembly can comprise an access septum, and the MEMS module can comprise a needle or other penetrating element for penetrating the access septum to provide a fluid pathway between the transcutaneous delivery assembly and the pumping mechanism. The needle or other penetrating element can comprise a needle (e.g. a 28 gauge needle) that extends at least 1 mm from the MEMS module. The transcutaneous delivery assembly can comprise a needle or other penetrating element, and the MEMS module can comprise an access septum for receiving the needle or other penetrating element to provide a fluid pathway between the transcutaneous delivery assembly and the pumping mechanism. The needle or other penetrating element can comprise a needle (e.g. a 28 gauge needle) that extends at least 1 mm from the transcutaneous delivery assembly. The MEMS module can comprise a silicon diaphragm actuated via micro-machined PZT film. The MEMS module can comprise: an actuator with three chambers, an input side and an output side; a first active valve configured to regulate flow on the input side; and a second active valve configured to regulate flow on the output side. The MEMS module can be configured to provide a stroke volume of 1.3 nL. The MEMS module can be configured to provide a stroke volume of no more than 15 nL. The MEMS module can comprise a chamber with a volume of 10.6 nL. The MEMS module can be configured to provide an output pressure of at least 60 kPa. The MEMS module can comprise a piezo drive element. The piezo drive element can comprise a volume defined by 2.1 mm×2.2 mm×0.6 mm.

In some embodiments, the transcutaneous delivery assembly comprises a skin-penetrating distal portion that includes a tip portion. The tip portion can be configured to be positioned within subcutaneous tissue of the patient. The tip portion can be configured to be positioned in a patient location selected from the group consisting of: subcutaneous tissue; an artery; a vein; a location proximate a nerve; a location proximate an organ; a muscle; a bone; adipose tissue; a location proximate an implanted access port; and combinations thereof. The delivery device can comprise an insertion assembly configured to advance the tip portion through the skin of the patient. The insertion assembly can be configured to advance the tip portion into the subcutaneous tissue of the patient.

In some embodiments, the transcutaneous delivery assembly comprises a needle and a surrounding cannula. The needle can comprise a metal needle. The cannula can comprise a cannula comprising one or more polymers. The cannula can comprise an internal metal coil. The cannula can comprise multiple outlets, and each outlet can be configured to deliver the first agent to the patient. The needle can be configured to be withdrawn from the patient after the cannula can be positioned within the patient. The needle can be configured to be automatically withdrawn from the patient after the cannula can be positioned within the patient.

In some embodiments, the delivery device further comprises at least one sensor. The at least one sensor can comprise a first sensor constructed and arranged to detect a parameter of a material of the delivery device. The first sensor can be configured to detect a parameter of the first agent. The at least one sensor can comprise a sensor constructed and arranged to detect a parameter of the patient. The at least one sensor can comprise a sensor constructed and arranged to detect a physiologic parameter of the patient selected from the group consisting of: glucose level; blood glucose level; a blood gas parameter; blood pressure; heart rate; an electrocardiogram (EKG); an electroencephalogram (EEG); a respiration parameter; pH; a hormone level; galvanic skin response (GSR) and/or electrodermal activity (EDA); and combinations thereof. The at least one sensor can comprise a bubble detector. The at least one sensor can comprise a particle detector. The particle detector can comprise an LED-based particle scattering detector. The at least one sensor can comprise a pressure sensor. Pressure sensor can be constructed and arranged to measure the pressure of fluid within the reservoir. The pressure sensor can comprise a range of 200 kPa to −200 kPa, and/or a sensitivity of 1 Pa. The pressure sensor can be configured to measure the pressure of fluid within the transcutaneous delivery assembly. The pressure sensor can comprise a range of 0 kPa to 5.3 kPa, and/or a sensitivity of 8 Pa. The at least one sensor can comprise a flow sensor. The flow sensor can comprise a platinum hot wire anemometer. The anemometer can comprise one heated wire and two unheated wires. The at least one sensor can comprise a temperature sensor.

In some embodiments, the delivery device further comprises a bubble capture element. The bubble capture element can be positioned proximate the reservoir.

In some embodiments, the system further comprises the first agent. The first agent can comprise an agent selected from the group consisting of: an agent configured to improve and/or maintain the health of a patient; a drug; a pharmaceutical drug; a hormone; a protein; a protein derivative; a small molecule; an antibody; an antibody derivative; an excipient; a reagent; a buffer; a vitamin; a nutraceutical; and combinations thereof. The first agent can be insulin and/or an analog of insulin (either or both, “insulin” herein). The first agent can be concentrated ultra-rapid insulin. The first agent can comprise insulin with a concentration of at least 100 Units/mL. The first agent can comprise insulin with a concentration of at least 200, 220, 300, or 500 Units/mL. The first agent can comprise glucagon and/or glucose. The first agent can comprise an agent selected from the group consisting of: insulin; an insulin analog; glucagon, glucose; dextrose; amylin; ghrelin; estrogen; progesterone; prolactin; testosterone; serotonin; cortisol; triiodothyronine; thyroxine; adrenaline; somatotropin; GCSF; HGF; and analogs and/or combinations thereof.

In some embodiments, the system further comprises a sensing device configured to measure a parameter. The parameter can comprise an environmental parameter of the patient and/or the delivery device. The environmental parameter can comprise a parameter selected from the group consisting of: ambient temperature; ambient pressure; ambient humidity; and combinations thereof. The parameter can comprise a physiologic parameter of the patient. The sensing device can be configured to monitor a patient parameter selected from the group consisting of: glucose level; blood glucose level; a blood gas parameter; blood pressure; heart rate; an electrocardiogram (EKG); an electroencephalogram (EEG); a respiration parameter; pH; a hormone level; galvanic skin response (GSR) and/or electrodermal activity (EDA); and combinations thereof. The parameter can comprise a glucose level of the patient. The sensing device can be configured to monitor the patient's glucose continuously. At least a portion of the sensing device can be positioned within the housing.

In some embodiments, the system further comprises a communication device. The communication device can comprise a device selected from the group consisting of: smart phone; cell phone; tablet; laptop computer; computer; personal data assistant; smart watch; special purpose communication device; and combinations thereof. The communication device can be configured to intermittently communicate with the delivery device. The delivery device can be configured to receive data from the delivery device, and the device can transfer the received data to a computer network. The computer network can be configured to perform a backup of the received data. The computer network can be configured to communicate the received data to a caregiver of the patient and/or a third-party payor related to the treatment of the patient. The network can be configured to perform cross-patient learning. The network can be configured to perform cross-patient machine learning.

In some embodiments, the system further comprises a fill device configured to fill the reservoir with the first agent. The fill device can be constructed to fill the reservoir with the first agent at a rate of at least 5 mL/min, 10 mL/min, 20 mL/min, 60 mL/min, or 120 mL/min.

In some embodiments, the system further comprises an insertion device, and the transcutaneous delivery assembly comprises a first skin-penetrating distal portion, and the insertion device is configured to advance the first skin-penetrating distal portion through the skin of the patient. The insertion device can comprise a needle for insertion through the skin-penetrating distal portion. The insertion device can be configured to insert the first skin-penetrating distal portion at a distance that can be adjustable. The insertion device can be configured to position the first skin-penetrating distal portion within subcutaneous tissue of the patient. The insertion device can be configured to removably attach to the delivery device. The delivery device can further comprise a glucose-sensing device that includes a second skin-penetrating distal portion, and the insertion device can be further configured to advance the second skin-penetrating distal portion through the skin of the patient. The insertion device can comprise a needle for insertion through the second skin-penetrating distal portion. The insertion device can be configured to position the second skin-penetrating distal portion within subcutaneous tissue of the patient. The insertion device can be configured to insert the first skin-penetrating distal portion and the second skin-penetrating distal portion simultaneously. The insertion device can be configured to insert the second skin-penetrating distal portion at a distance that can be adjustable. The insertion device can be configured to electrically connect the glucose sensing device and the control module. The insertion device can be configured to make the electrical connection during insertion of the second skin-penetrating distal portion. The insertion device can be configured to create a seal between the glucose sensing device and the housing. The insertion device can be configured to create the seal during insertion of the second skin-penetrating distal portion.

According to another aspect of the present inventive concepts, a method of delivering an agent to a patient comprises: providing a delivery system and delivering a first agent to the patient.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates a schematic view of a system for delivering one or more agents to a patient, consistent with the present inventive concepts.

FIG. 2A illustrates a schematic view of a system for delivering at least two agents to a patient, consistent with the present inventive concepts.

FIG. 2B illustrates a schematic view of another system for delivering one or more agents to a patient, consistent with the present inventive concepts.

FIGS. 3A-B illustrate side and top sectional views, respectively, of a delivery device, consistent with the present inventive concepts.

FIGS. 4A-E illustrate various views of a reservoir, reservoir analysis, and a reservoir-forming tool are illustrated, consistent with the present inventive concepts.

FIG. 5 illustrates a sectional transparent view of a portion of a delivery device, consistent with the present inventive concepts.

FIGS. 6A-C illustrate sequential views of a procedure for filling a reservoir, consistent with the present inventive concepts.

FIGS. 7A-E illustrate views of a procedure for priming a delivery device and introducing an agent delivery cannula into a patient using an insertion device, consistent with the present inventive concepts.

FIGS. 8A-E illustrate views of a procedure for introducing an agent delivery cannula into a patient using an insertion device, consistent with the present inventive concepts.

FIGS. 9A-E illustrate views of a procedure for introducing an agent delivery cannula and a sensing assembly into a patient using an insertion device, consistent with the present inventive concepts.

FIGS. 10A-B illustrate various views of a pumping mechanism, consistent with the present inventive concepts.

FIG. 11 illustrates a flow chart for manufacturing a pumping mechanism, consistent with the present inventive concepts.

FIG. 12 illustrates a fluidic connection layer including a substrate, consistent with the present inventive concepts.

FIG. 13 illustrates a sectional and a magnified view of a portion of a pumping mechanism including a microfluidic routing layer, consistent with the present inventive concepts.

FIG. 14 illustrates a perspective and a magnified sectional view of a fluid path connecting element, consistent with the present inventive concepts.

FIGS. 15A-D illustrate a pumping actuator schematic, a series of manufacturing steps for a pumping actuator, a set of three pumping actuator views, and a series of pump activation steps, respectively, consistent with the present inventive concepts.

FIGS. 16A-B illustrate results of a finite element analysis (FEA) on a pumping mechanism, consistent with the present inventive concepts.

FIGS. 17A-B illustrate a perspective view and an assembly process, respectively, of an embodiment of a flow sensor, consistent with the present inventive concepts.

FIGS. 18A-E illustrate various views of an embodiment of a passive check valve, consistent with the present inventive concepts.

FIGS. 19A-D illustrate various views of an embodiment of an active check valve, consistent with the present inventive concepts.

FIG. 20 illustrates a side view of a cannula, consistent with the present inventive concepts.

FIG. 21 illustrates a schematic view of a communication architecture for an agent delivery system, consistent with the present inventive concepts.

FIG. 22 illustrates a schematic view of a delivery device, consistent with the present inventive concepts.

FIG. 23 illustrates a schematic view of a delivery system and a flow chart of a learning architecture for the delivery system, consistent with the present inventive concepts.

FIG. 24 illustrates a schematic view of a delivery system, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

It will be understood that the words “comprising” (and any form of comprising, such as “comprise” and “comprises”), “having” (and any form of having, such as “have” and “has”), “including” (and any form of including, such as “includes” and “include”) and/or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of two or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, “prevention” and the like, where used herein, shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. “Positive pressure” includes pressure above room pressure or simply a pressure that is greater than another pressure, such as a positive differential pressure across a fluid pathway component such as a valve. “Negative pressure” includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “transducer” is to be taken to include any component or combination of components that receives energy or any input and produces an output. For example, a transducer can include an electrode that receives electrical energy and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: heat energy to tissue; cryogenic energy to tissue; electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of two or more of these. A transducer can include a component configured to neutralize an ablative process, such as a transducer configured to cool tissue prior to and/or after a heat ablation of tissue, and/or a transducer configured to warm tissue prior to and/or after a cryogenic ablation of tissue. Alternatively or additionally, a transducer can comprise a mechanism, such as: a valve; a grasping element; an anchoring mechanism; an electrically-activated mechanism; a mechanically-activated mechanism; and/or a thermally activated mechanism.

As used herein, the term “functional element” is to be taken to include one or more elements constructed and arranged to perform a function. A functional element can comprise one or more sensors and/or one or more transducers. In some embodiments, a functional element is configured to deliver energy and/or otherwise treat tissue (e.g. a functional element configured as a treatment element). Alternatively or additionally, a functional element (e.g. comprising one or more sensors) can be configured to record one or more parameters, such as a patient physiologic parameter; a patient anatomical parameter (e.g. a tissue parameter); a patient environment parameter; and/or a system parameter (e.g. temperature and/or pressure within the system). In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a patient anatomical parameter; and combinations of two or more of these. A functional element can comprise a fluid, such as an ablative fluid (as described herein) comprising a liquid, gel, and/or gas configured to ablate or otherwise treat tissue. A functional element can comprise a reservoir, such as an expandable balloon configured to receive an agent. A “functional assembly” can comprise an assembly constructed and arranged to perform a function, such as is described hereabove. In some embodiments, a functional assembly is configured to deliver energy and/or otherwise treat tissue (e.g. a functional assembly configured as a treatment assembly). Alternatively or additionally, a functional assembly can be configured to record one or more parameters, such as a patient physiologic parameter; a patient anatomical parameter; a patient environment parameter; and/or a system parameter. A functional assembly can comprise an expandable assembly. A functional assembly can comprise one or more functional elements.

As used herein, the term “agent” shall include but not be limited to one or more agents selected from the group consisting of: an agent configured to improve and/or maintain the health of a patient; a drug (e.g. a pharmaceutical drug); a hormone; a protein; a protein derivative; a small molecule; an antibody; an antibody derivative; an excipient; a reagent; a buffer; a vitamin; a nutraceutical; and combinations of these.

It is an object of the present inventive concepts to provide systems, devices and methods for delivering one or more agents to a patient. The system includes a delivery device for delivering one or more drugs or other agents to a patient, such as insulin and/or an insulin analog. The delivery device can include one or more reservoirs for storing the one or more agents. A pumping mechanism delivers the agents into the patient, via a transcutaneously inserted cannula. The pumping mechanism can include a pump comprising a micro-electromechanical system (MEMS) pumping element. An assembly of the delivery device or a sensing device of the system can measure one or more physiologic parameters of the patient (e.g. glucose level), such as when this information is used to control (e.g. in a closed-loop mode) and/or otherwise impact the delivery of the agents.

The systems of the present inventive concepts can include lightweight, wearable delivery systems that provide numerous advantages. These agent delivery systems can be configured to provide telemetry data to caregivers that is useful to the caregiver in treating the patient, and these systems can also provide information that is useful in justifying reimbursement for treatment by insurance companies and other payors.

The systems of the present inventive concepts can be configured to deliver insulin and/or one or more other therapeutic agents. Insulin is a protein hormone that participates in glucose homeostasis by instructing a patient's cells to absorb glucose. Insulin analogs are proteins that can perform the same function as insulin in glucose metabolism. Patients with diabetes have insufficient insulin to prevent elevated blood glucose levels and so often must self-administer insulin analogs. Currently available pump-appropriate insulin analog formulations are unstable and so are not rated to be stored at ambient temperature (e.g. room temperature) for more than 30-45 days. There is a need for improved insulin analog delivery systems, devices, and methods.

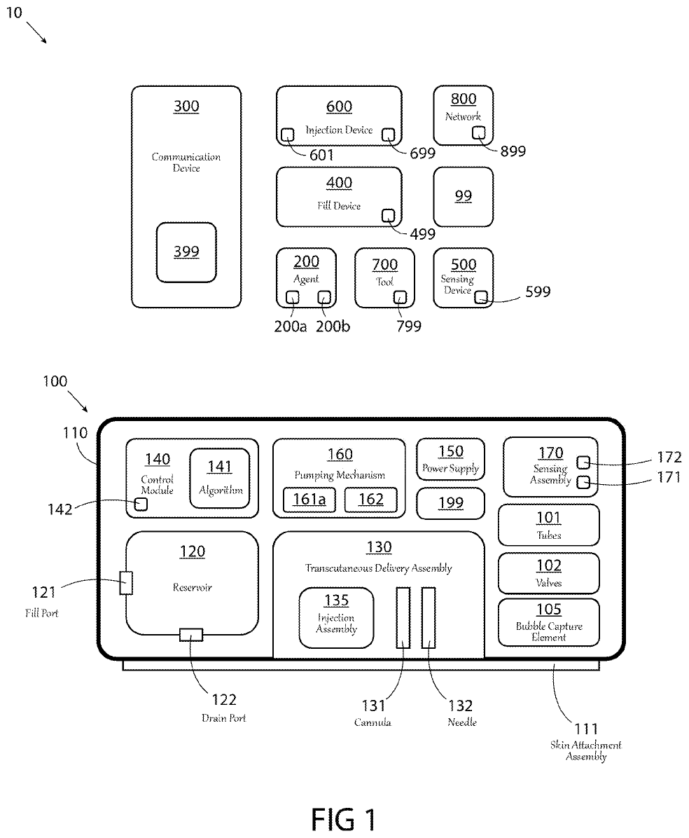

Referring now to FIG. 1, a schematic view of a system for delivering one or more agents to a patient is illustrated, consistent with the present inventive concepts. System 10 includes an agent delivery device, device 100 shown. In some embodiments, system 10 includes the one or more agents to be delivered by device 100, such as agent 200 shown. System 10 can include other components, such as communication device 300, fill device 400, glucose sensing device 500, insertion device 600, tool 700, and/or network 800, each as described in detail herein. One or more operators can interface with (e.g. control or configure) system 10, such as the patient, a member of the patient's family, and/or a caregiver of the patient (e.g. a doctor or nurse).

Delivery device 100 can comprise various fluidic, mechanical, and/or electronic components. Delivery device 100 can comprise: one or more housings, housing 110; one or more reservoirs for storing a fluid, reservoir 120; one or more transcutaneous delivery assemblies for delivering fluid to an internal body location of the patient, TDA 130; a module for controlling operation of delivery device 100 components, control module 140; one or more supplies of energy, power supply 150; one or more assemblies for propelling fluid, pumping mechanism 160; and/or one or more assemblies for measuring a patient parameter (e.g. a patient environment and/or a patient physiologic parameter), sensing assembly 170; each as shown in FIG. 1 and described herein.

Delivery device 100 can comprise one or more housings, housing 110 shown. Housing 110 can comprise rigid materials, flexible materials, or both. In some embodiments, housing 110 comprises a component for attaching (e.g. removably attaching) device 100 to the patient's skin, skin attachment assembly 111. Skin attachment assembly 111 can include an adhesive surface, such as an adhesive surface that is protected via a removable cover layer prior to attachment to the patient's skin.

Delivery device 100 can comprise one or more conduits, tubes 101, such as for fluidly connecting two or more components of device 100.

Delivery device 100 can comprise one or more valves, valves 102 shown.

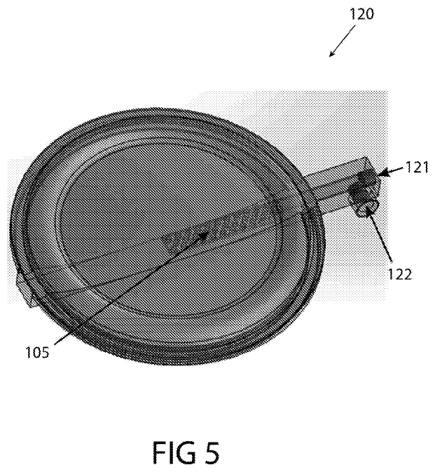

Delivery device 100 can comprise one or more elements for capturing or removing bubbles from a flow path of device 100, bubble capture element 105. Bubble capture element 105 can be constructed and arranged as described in reference to FIG. 5 herein. Bubble capture element 105 can be integrated into reservoir 120 (e.g. integrated into a wall portion of reservoir 120).

Housing 110 can comprise one or more openings, such as: an access hole for filling reservoir 120; an opening through which one or more cannulas may pass through into the patient's skin (e.g. cannula 131 and/or 171 described herein).

In some embodiments, delivery device 100 (e.g. housing 110) comprises a major axis of no more than 45 mm, 40 mm, 35 mm, or 33 mm, and a height of no more than 10 mm, 8 mm, 7.5 mm, or 6.5 mm.

Delivery device 100 may include a draining system for removal of fluid (e.g. water) that may enter housing 110 when the patient is exposed to water. In some embodiments, one or more internal portions (e.g. electronics) of delivery device 100 can include a water-resistant coating, such as is described herein. In these embodiments, delivery device 100 may be constructed and arranged to allow patient fluids (e.g. sweat) and/or environmental fluids (e.g. bathing fluids) to pass through one or more internal portions of housing 110 without adverse effect. Housing 110 can include one or more openings positioned to allow proper drainage (e.g. using gravity) when positioned on the patient in a particular orientation.

Housing 110 can comprise a transparent portion that is positioned above a similar transparent portion in reservoir 120, such that the amount and/or condition of fluid present in reservoir 120 can be observed by the patient or other operator of system 10.

Delivery device 100 can comprise a fluid storage component, reservoir 120 shown, such as for storing agent 200. Reservoir 120 can comprise a circular or at least relatively circular geometry (“circular geometry” herein). Reservoir 120 can comprise a port, fill port 121, for delivering agent 200 or other fluid into reservoir 120 (e.g. in an initial filling procedure and/or in a re-filling procedure). Reservoir 120 can comprise another port, drain port 122, for fluid to exit reservoir 120. Reservoir 120 can be provided (e.g. provided to the patient) pre-filled with agent 200, such as when agent 200 comprises a stabilized formulation of insulin, glucagon, glucose, and/or dextrose. In some embodiments, reservoir 120 is pre-filled with agent 200 (e.g. insulin), and stored for a period of at least 90 days, at least 120 days, at least 150 days, at least 180 days, at least 270 days, and/or at least 365 days prior to use by the patient (e.g. prior to insertion of a cannula, cannula 131 described herein, into the patient), such as when agent 200 comprises insulin. In some embodiments, one or more reservoirs 120 can be maintained in a controlled environment, such as an environment maintained at a temperature of 4° C., 10° C., 20° C., 25° C., 30° C., or 37° C. In some embodiments, one or more reservoirs 120 are stored at a temperature that varies over time. In some embodiments, reservoir 120 comprises a penetrable input and/or output port, such as a port comprising a septum, valve, or other penetrable component configured to provide access (“septum” or “access septum” herein). Alternatively, reservoir 120 can comprise an input and/or output port with a needle or other penetrating element (“needle” herein), such as a needle configured to penetrate a septum, such as an access septum of fill device 200 and/or pumping mechanism 160.

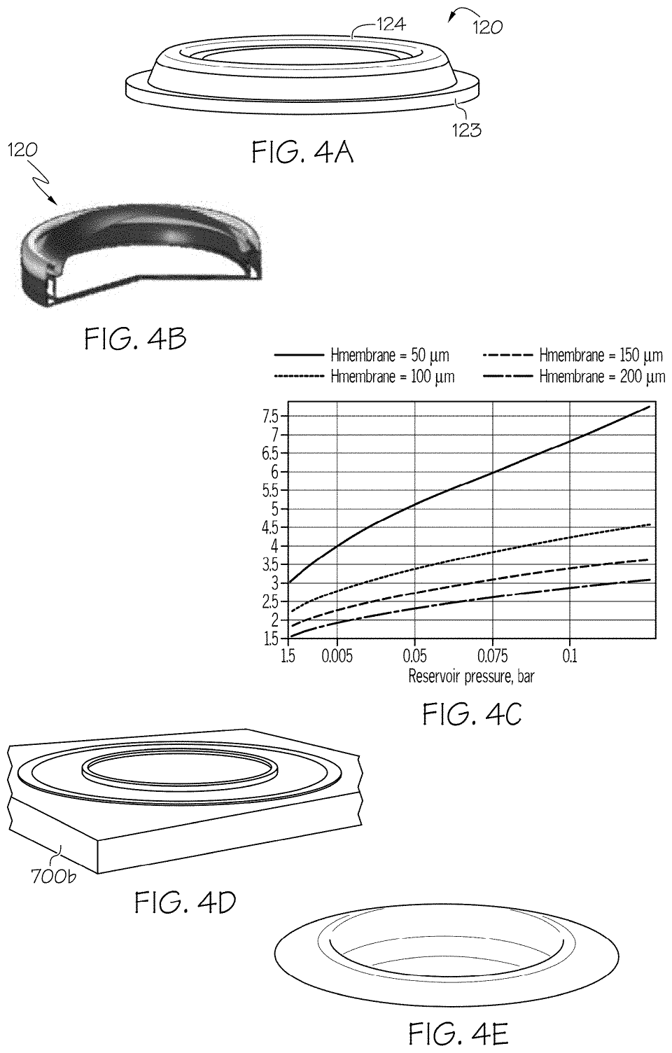

Reservoir 120 can comprise a solid portion and a film portion (e.g. a flexible film portion arranged in a “rolling sock” configuration as described in reference to FIGS. 4A-E), either or both of which can be positioned to contact agent 200. Alternatively or additionally, reservoir 120 can comprise an assembly including two flexible films that are bonded together. In some embodiments, reservoir 120 comprises a film that is flexible but resists stretching (e.g. resist stretching in at least one direction). In some embodiments, reservoir 120 comprises a film that includes at least a portion that is configured to stretch. The solid and film portions can be bonded together using: ultrasound, photochemical cross-linking, adhesive, and/or heat. The film portion can comprise a multi-layer construction, where different layers are configured to provide different functions, such as one layer with a low permeability (e.g. to a solvent) and compatibility with a particular agent (e.g. insulin or glucagon), and a different layer with a low permeability to oxygen and/or water. The solid portion can comprise one or more ports, such as a port located on the side of reservoir 120 and/or a port located on the bottom of reservoir 120.

Reservoir 120 can comprise a rigid base, and an attached flexible film (e.g. as described in reference to FIG. 4A-E herein).

Reservoir 120 can be configured to undergo one or more movements (e.g. rotations) relative to at least a portion of housing 110, as described herein. These movements can cause (e.g. automatically cause): one or more fluid connections to be made (e.g. a fluid connection between reservoir 120, pumping mechanism 160, and/or TDA 130, each as described herein); a priming procedure to be performed (e.g. a fluid pathway in and/or between a delivery device 100 component to be primed); an “on state” to be entered (e.g. delivery device 100 to turn on); and/or another device 100 function to be performed (e.g. as configured by algorithm 141).

In some embodiments, delivery device 100 weighs less than 10 grams, or less than 8 grams, when full with agent 200. In some embodiments, reservoir 120 can comprise a fill volume of no more than 0.1 mL, 0.2 mL, 0.5 mL, 0.8 mL, 1.0 mL, 1.2 mL, 1.5 mL, or 2.4 mL.

Delivery device 100 can comprise TDA 130, which can comprise an assembly configured to deliver agent 200 to the patient. Assembly 130 can comprise one or more fluid delivery tubes, cannula 131, for insertion through the skin of the patient such as to deliver agent 200 to the patient. In some embodiments, cannula 131 comprises metal or polytetrafluoroethylene (PTFE). In some embodiments, cannula 131 comprises a flexible material, and cannula 131 surrounds a rigid needle, needle 132. In some embodiments, insertion device 600, as described herein, comprises needle 132. In some embodiments, assembly 130 comprises an assembly for inserting cannula 131 through the patient's skin, insertion assembly 135 shown. Insertion assembly 135 can be integrated with other components of device 100, such as when assembly 135 is positioned within housing 110. Alternatively or additionally, system 10 can include a separate device for inserting cannula 131 through the patient's skin, insertion device 600 described herein.

Cannula 131 can comprise a soft polymeric (e.g. vinyl) shaft. In some embodiments, cannula 131 comprises a braided construction (e.g. includes a metal or polymeric reinforcing coil). Cannula 131 can comprise a distal portion, including a distal-most tip portion. The tip portion can be configured to be positioned at one or more internal body locations, such as a location selected from the group consisting of: subcutaneous tissue; an artery; a vein; a location proximate a nerve; a location proximate an organ; a muscle; a bone; adipose tissue; a location proximate an implanted access port; and combinations thereof. The tip portion of cannula 131 can comprise an end hole, and/or one or more side holes (e.g. 3 side holes), for delivery of agent 200 into the patient. Cannula 131 can comprise a length of between 3 mm and 8 mm. Cannula 131 can comprise a coating, such as an antimicrobial coating (e.g. a silver and/or silver salt coating), an anti-inflammatory coating (e.g. a corticosteroid), an electrically conductive coating, and/or a lubricious coating (e.g. a silicon oil to ease in insertion into the patient). In some embodiments, cannula 131 comprises an integrated sensor, such as an integrated sensor-based functional element 199 comprising a glucose or other physiologic sensor.

TDA 130 can be configured to provide or maintain a seal between cannula 131 and housing 120 during insertion of cannula 131 into the patient.

TDA 130 can comprise a septum and/or a needle, for fluidly connecting to a needle and/or septum, respectively, of pumping mechanism 160 (e.g. a fluid connection provided via rotation, as described herein). In some embodiments, TDA 130 comprises a septum that is configured to be penetrated in two directions, such as in a first direction by a needle of pumping mechanism 160, and in a second direction by needle 132 (e.g. needle 132 of TDA 130 or of insertion device 600). In some embodiments, needle 132 passes through the septum and through the lumen of the cannula 131 to facilitate insertion of the cannula 131 into the body. In these embodiments, pumping mechanism 160 can be isolated from contamination by the insertion device 600 (e.g. by the septum).

While delivery device TDA 130 is described as an assembly including at least one cannula 131 that is inserted through the patient's skin, it should be considered within the spirit and scope of this application that TDA 130 can alternatively (or additionally) include other routes of delivery for agent 200, such as when TDA 130 comprises a connecting element configured to attach to a separate transcutaneous device (e.g. a transcutaneous catheter or infusion set such as an IV infusion set), or when TDA 130 comprises a transdermal agent delivery element, such as an element configured to cause agent 200 to pass through the patient's skin to an internal body location (e.g. via electrical fields that drive agent 200 through the patient's tissue).

Delivery device 100 can comprise an electronic assembly, control module 140, which can comprise one or more assemblies and/or discrete components that are configured to provide control signals, record data, and/or perform other functions of device 100. Control module 140 can comprise one or more algorithms, algorithm 141 shown. Control module 140 can further comprise various electronic componentry, electronics 142 shown. Electronics 142 can comprise various electronic assemblies and other electronic components, such as components selected from the group consisting of: a microprocessor; a microcontroller; electronic memory; control circuitry; MEMS control circuitry; signal conditioning circuitry; switching circuitry; sensor interface circuitry; power supply circuitry; communication circuitry (e.g. Bluetooth communication circuitry); and combinations of these.

Control module 140 can include electronic hardware components and embedded software. Control module 140 can comprise a component selected from the group consisting of: a printed circuit board (PCB); a microprocessor; input and/or output circuitry; one or more sensor-based functional element 199 configured to measure patient and/or device 100 motion; an alarm element (e.g. functional element 199 configured as an audible, visible, and/or tactile alarm); and combinations thereof. Control module 140 can comprise a PCB that has an approximate disk shape with an approximate annulus arc extension.

Control module 140, and other portions of delivery device 100, can include electronic hardware that comprises a coating configured to provide a fluid-resistance (e.g. prevent or at least reduce egress of fluid to electronic components, such as integrated circuits, or electronic filaments. such as wires or conductive traces).

Algorithm 141 can be configured to adjust and/or otherwise control the delivery (“adjust the delivery” or “control the delivery” herein) of agent 200. Algorithm 141 can comprise a learning algorithm, such as a machine learning algorithm. In some embodiments, algorithm 141 comprise a learning algorithm that is performed, at least partially, in the cloud, as described herein (e.g. via a wired or wireless connection). Algorithm 141 can comprise a learning algorithm that determines optimal values for various parameters, such as parameters used by algorithm 141. Algorithm 141 can comprise an algorithm based on and/or otherwise analyzing information related to the patient's glucose levels and/or other patient physiologic parameter.

Algorithm 141 can be configured to adjust the delivery of agent 200 based on one or more sensors that measure a parameter of delivery device 100, such as a functional element 199 comprising a temperature and/or pressure sensor whose measurements impact delivery of agent 200 by device 100.

Alternatively or additionally, algorithm 141 can be configured to adjust the delivery of agent 200 based on one or more sensors that measure a parameter of the patient (e.g. in a closed-loop mode, such as when system 10 is configured as an artificial pancreas for a diabetic patient). For example, algorithm 141 can adjust the delivery of agent 200 based on a parameter selected from the group consisting of: glucose level or other patient physiologic parameter; patient motion and/or other patient activity; patient heart rate; patent galvanic skin resistance (GSR) and/or electrodermal activity (EDA); and combinations of these. In some embodiments, algorithm 141 can adjust the delivery of agent 200 based on a patient-disclosed plan (e.g. a meal plan and/or an exercise or other activity plan). Algorithm 141 can receive the delivery-determining data from sensing assembly 170, sensing device 500, and/or another sensor-based component of system 10. Algorithm 141 can receive delivery-determining data from the patient (e.g. via communication device 300), such as data regarding a planned meal for the patient or planned activity of the patient, as described hereabove. Algorithm 141 can be customized based on the individual patient receiving agent 200 from the particular delivery device 100 that includes algorithm 141. The algorithm 141 can comprise a learning algorithm, such as learning related to the patient's response to agent 200 (e.g. insulin) delivery. Agent 200 delivery parameters used and/or determined by algorithm 141 can be uploaded to and/or downloaded from another component of system 10, such as communication device 300 and/or network 800.

In some embodiments, algorithm 141 shapes a flow delivery profile (e.g. a bolus of agent 200 to be delivered) based on a pressure (e.g. a pressure response) that is measured proximate TDA 130 (e.g. measured by a sensor-based functional element 199 positioned to measure pressure in a flow path within and/or otherwise proximate cannula 131).

In some embodiments, algorithm 141 is configured to cause system 10 (e.g. device 100) to enter an alarm state, such as when a functional element of system 10 comprises an audible, visible, and/or tactile transducer that alerts the patient when the alarm state is entered. For example, system 10 can enter an alarm state when one or more different system parameters (e.g. as measured by a sensor of system 10) and/or patient parameters (e.g. as measured by sensing assembly 170 or sensing device 500) exceeds a threshold.

In some embodiments, algorithm 141 regulates the delivery of agent 200 by delivery device 100, such as when algorithm 141 delivers agent 200 based on a measurement of a physiologic parameter of the patient (e.g. as provided by sensing assembly 170 and/or sensing device 500). For example, the algorithm 141 can receive glucose level information of the patient, and regulate insulin delivery based on the information. In these embodiments, if data regarding the physiologic parameter is unavailable (e.g. due to a malfunction or lack of availability), algorithm 141 can be configured to calculate an estimated delivery profile, but cause an amount less than the estimated amount to be delivered to the patient (e.g. when algorithm 141 comprises a bias toward under-delivery as described herebelow).

In some embodiments, algorithm 141 receives data from multiple sources (e.g. similar or different types of data), and it delivers agent 141 based on an analysis of the data from the multiple sources. For example, algorithm 141 can receive data comprising: long term patient glucose level data; delivery device 100 flow data; delivery device 100 pulse count data; and/or other data. In some embodiments, algorithm 141 receives data from at least the cloud.

In some embodiments, algorithm 141 is configured to compare a measured pressure of agent 200 and compare it to an expected level of that pressure. Algorithm 141 can receive pressure information from a pressure-sensor based functional element 199 positioned proximate a flow path containing agent 200 (e.g. proximate a flow path of pumping mechanism 160 and/or TDA 130). If the difference between the measured pressure and the expected pressure exceeds a threshold, algorithm 141 can be configured to: adjust pumping mechanism 160; and/or cause delivery device 100 to enter an alarm state.

In some embodiments, algorithm 141 comprises a bias where algorithm 141 causes less than a target level of agent 200 to be delivered, “under-delivery” herein, or a bias where algorithm causes more than a target level of agent 200 to be delivered, “over-delivery” herein. For example, algorithm 141 can comprise a bias configured to cause delivery device 100 to avoid over-delivery of insulin or other agent 200 (e.g. to intentionally cause under-delivery of agent 200 if a failure mode is present, and/or data used by system 10 is suspected to be inaccurate or corrupted). Alternatively, such as when under-delivery of agent 200 creates a more serious adverse event than over-delivery, algorithm 141 can comprise a bias to avoid under-delivery of agent 200.

Delivery device 100 can comprise power supply 150, which can include one or more batteries, capacitors, and/or other sources of energy. Power supply 150 can supply energy to one or more components of device 100, such as control module 140, pumping mechanism 160, functional element 199 (described herebelow), and/or other components of device 100. In some embodiments power supply 150 comprises a rechargeable supply (e.g. a rechargeable battery and/or capacitor), such as when delivery device 100 is configured to wirelessly recharge power supply 150. In some embodiments, power supply 150 comprises a component (e.g. a battery and/or capacitor) with a relatively flat geometry, such as a relatively flat component that is positioned under or above reservoir 120 (e.g. a reservoir 120 with a relatively flat geometry as well). In some embodiments, power supply 150 comprises a supply configured to supply at least 50 joules, such as at least 100, 500, 1,000, and/or 2000 joules of energy, such as when power supply 150 is configured to provide 1,080 joules of energy. In some embodiments, power supply 150 is configured to provide energy to delivery device 100 to support operation of delivery device 100 for at least 3 days, 3.3 days, 5.3 days, 7 days, 7.3 days, or 11.3 days. In some embodiments, power supply 150 comprises a slidable insulator configured to electrically isolate power supply 150 from one or more (e.g. all or a subset of) electronic components of delivery device 100 (e.g. isolate power supply 150 from control module 140). In these embodiments, an operator can pull out or otherwise move the slidable insulator (e.g. an insulator in a tear-tab arrangement), such as to electrically connect power supply 150 to the previously non-connected electronic components of delivery device 100. In some embodiments, one or more electronic components are electrically connected to power supply 150 (e.g. a clock circuit) when the slidable insulator is in place, and the remainder of electronic components of delivery device 100 become electrically connected when the slidable insulator is moved.

Delivery device 100 can comprise pumping mechanism 160, which can comprise a fluidic assembly and/or other assembly including one or more components configured to propel agent 200 (and/or other fluids) through flow paths of device 100 and into the patient. Pumping mechanism 160 can comprise one or more pumping elements, pump 161, such as a MEMS pumping element. Alternatively or additionally, pump 161 can comprise a pumping element selected from the group consisting of: a syringe pump; a displacement pump; a peristaltic pump; and combinations of these. Pumping mechanism 160 can be configured to provide fluid (e.g. agent 200) continuously and/or in discrete boluses. For example, these boluses provided by pump 161 can comprise a volume of no more than 1 nL, 2 nL, 5 nL, 10 nL, 15 nL, 25 nL, or 50 nL. In some embodiments, pump 161 comprises a MEMS pump and the bolus volume is at least 10 nL, 15 nL, 20 nL, or 25 nL.

Pumping mechanism 160 can comprise a fluidic connection layer, connection layer 162, such as a microfluidic connection layer. Connection layer 162 can provide fluid pathways between one or more components of pumping mechanism 160 and/or other components of delivery device 100.

In some embodiments, pump 161 comprises a pumping diaphragm, diaphragm 16017 described herein, (e.g. a piezoelectric film) constructed of a material selected from the group consisting of: lead zirconate titanate (PZT); aluminum nitride (AN); scandium doped aluminum nitride (ScAIN); and combinations of these.

In some embodiments, pump 161 comprises a piezo actuator drive circuit, such as the Boreas Technologies BOS1901 circuitry, such as a drive circuit with dimensions of 2.1 mm by 2.2 mm by 0.6 mm.

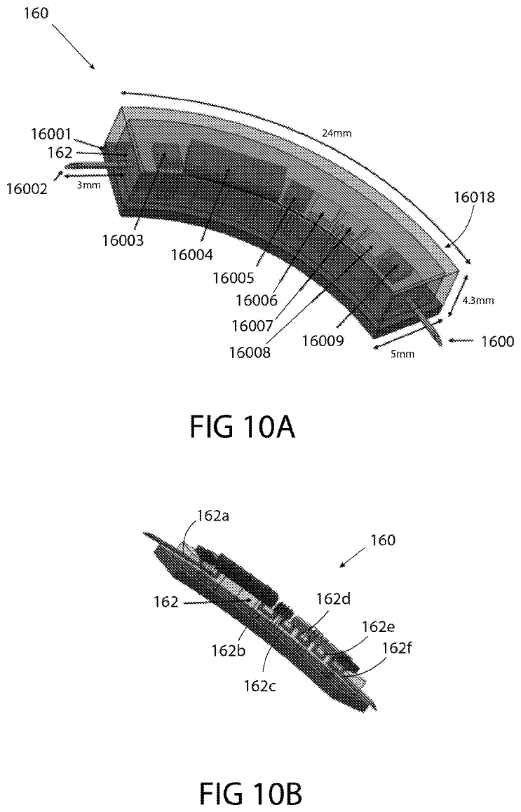

Pump 161 can comprise a MEMS pump including a composite of one or more silicon sensors and actuator that are assembled (e.g. microassembled) on top of a microfluidic substrate, as described in reference to FIGS. 10A-B herein. Pump 161 can comprise a MEMS pump including multiple chambers.

Pump 161 (e.g. a MEMS pump) can be constructed and arranged to deliver a stroke volume of no more than 50 nL. In some embodiments, pump 161 comprises a MEMS pump and the stroke volume is at least 10 nL, 15 nL, 20 nL, or 25 nL. Pump 161 can comprise a MEMS pump that is constructed and arranged to produce a pressure of at least 200 mmHg.

Pumping mechanism 160 can include one or more sensors and/or one or more valves, as described herein.

Pumping mechanism 160 can include one or more needles, such as a needle for penetrating a septum of reservoir 120 and/or a needle for penetrating a septum of TDA 130. Alternatively, either or both reservoir 120 or assembly 130 can comprise the needle, and pumping mechanism 160 can comprise a receiving septum. The needles used can comprise a beveled needle, such as a needle with a five-edge bevel. The needles can be made of surgical steel, can comprise a length of at least 3 mm, or at least 5 mm (e.g. extend at least 1 mm from a surface), and/or can have a diameter of at least 30Ga, such as a diameter of 28Ga.

Connecting layer 162 can provide pathways to fluidly connect various MEMS elements of pump 161 and/or other portions of pumping mechanism 160. The fluid pathways of pumping mechanism 160, and other fluid pathways of delivery device 100 and system 10, can include beveled edges, filleted corners (e.g. corners 16012 described herein) and/or other features configured to reduce shear forces, reduce turbulent flow, and/or otherwise enhance flow dynamics to reduce potential damage to agent 200 (e.g. insulin). The fluid pathways can comprise a feature such as a projection or reduced diameter portion configured to prevent undesired motion of an inserted needle or other tube.

Pumping mechanism 160 can include an electronics packaging system comprising at least one of a base, one or more wire-bond connections, a cover, an epoxy fill and/or a fluorosilicone gel fill. The electronics packaging system of pumping mechanism 160 can be in physical contact with an electronics assembly of control module 140. The electronics packaging system can be attached to a printed circuit board of the control module 140, such as when attached to an annulus arc extension of the PCB.

Pumping mechanism 160 can comprise a curved geometry, such as is described herein, such as a geometry in the shape of an annulus arc (e.g. an annulus arc with a radius of no more than 2 cm). In some embodiments, pumping mechanism 160 is positioned proximate the periphery of housing 110, also as described herein.

Delivery device 100 can comprise an integrated assembly for sensing a patient parameter, sensing assembly 170 shown. Sensing assembly 170 can be configured to measure a patient physiologic parameter and/or a patient environment parameter (e.g. a measure of the temperature, pressure, humidity, and/or other parameter of the room or other environment in which the patient is currently present). Sensing assembly 170 can comprise one or more filaments for measuring a patient physiologic parameter, cannula 171. Cannula 171 can be configured for insertion into the patient's skin. In some embodiments, cannula 171 surrounds a needle, needle 172, to aid in insertion of cannula 171 through the patient's skin. Needle 172 can be retracted soon after penetration, similar to the retraction of needle 132 from cannula 131, each as described herein. In some embodiments, insertion device 600, as described herein, comprises needle 172. Sensing assembly 170 can comprise one or more sensors and/or other components such that sensing assembly 170 can be configured to measure one or more parameters of a patient. Sensing assembly 170 can be configured to measure a glucose level of the patient (e.g. a blood glucose level). In some embodiments, sensing assembly 170 is configured to measure a patient parameter selected from the group consisting of: glucose level; blood glucose level; a blood gas parameter; blood pressure; heart rate; an electrocardiogram (EKG); galvanic skin resistance (GSR) and/or electrodermal activity (EDA); an electroencephalogram (EEG); a respiration parameter; pH; a hormone level; and combinations of these. In some embodiments, sensing assembly 170 is configured to measure a parameter of the environment of delivery device 100 and/or the patient, such as a parameter selected from the group consisting of: ambient temperature (e.g. room temperature); ambient pressure (e.g. room pressure); ambient humidity (e.g. room humidity); and combinations of these.

In some embodiments, sensing assembly 170 comprises two or more sensing devices, such as a first sensing assembly 170a and a second sensing assembly 170b. In these embodiments, data provided by both sensing assemblies 170a and 170b (e.g. similar and/or dissimilar patient physiologic and/or patient environment data) can be used by algorithm 141 to adjust and/or otherwise control delivery of agent 200 to the patient.

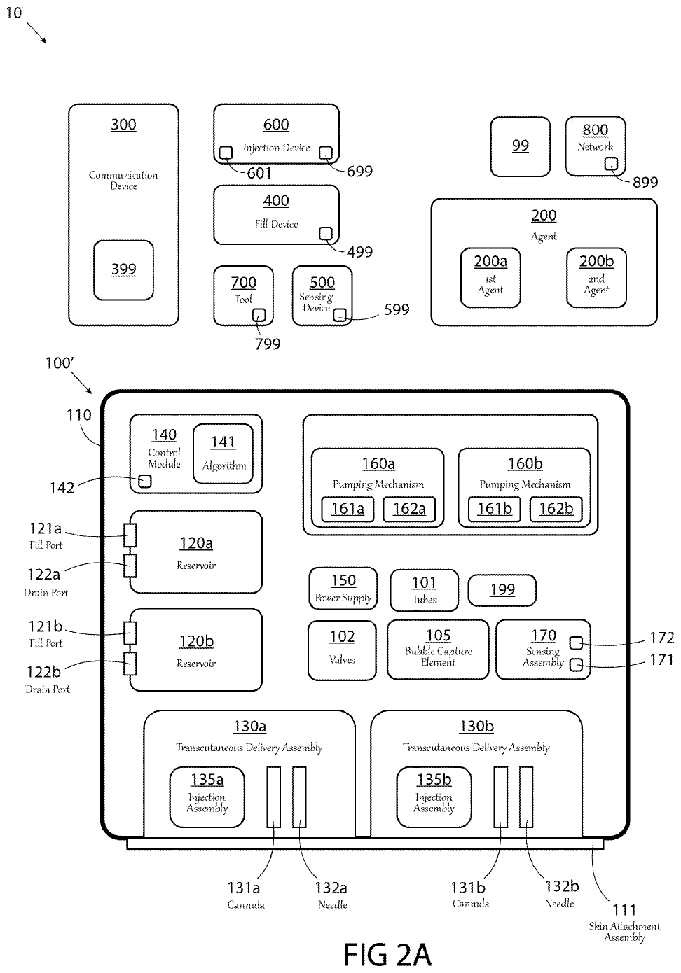

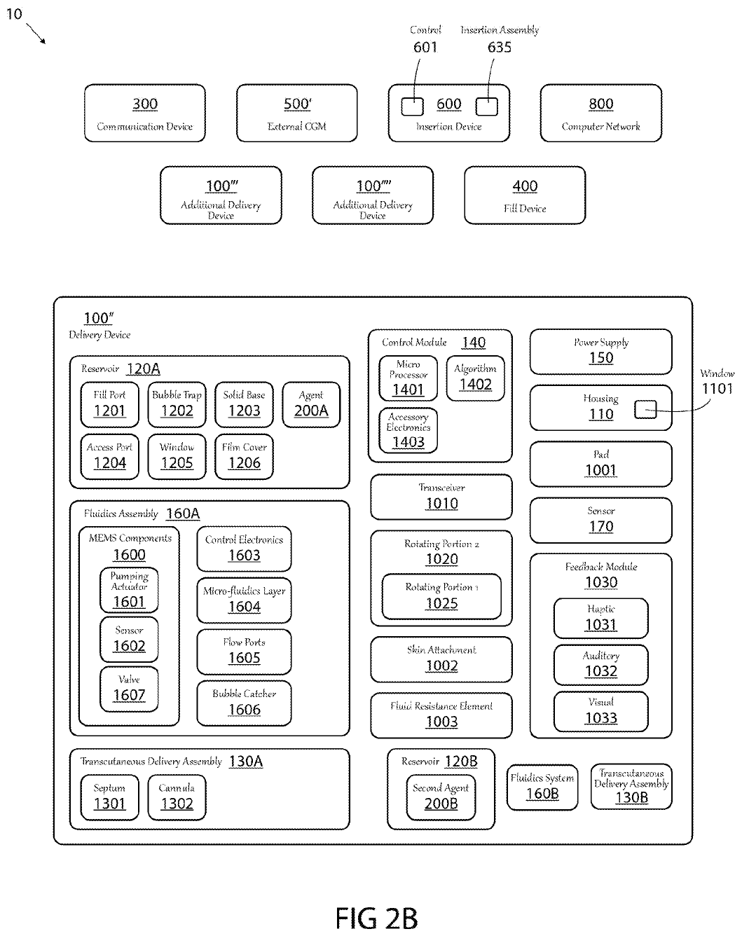

In some embodiments, delivery device 100 is configured to store (e.g. independently store) and deliver (e.g. independently deliver) at least two separate agents 200, such as delivery device 100′ of FIG. 2A and/or device 100″ of FIG. 2B described herein.

The various components of delivery device 100 can be connected with various conduits, such as tubes 101. These conduits are not shown for illustrative clarity, however include wires (e.g. single conductor, multiple conductor, and/or shielded wires), optical fibers, fluid delivery tubes (e.g. tubes 101), cables, linkages, wave guides, and the like. These conduits operably connect the various components, such as to transfer between two or more components: energy (e.g. energy provided by power supply 150); signals (e.g. signals provided by and/or analyzed by control module 140), data (e.g. data provided by and/or analyzed by control module 140), fluids (e.g. agent 200), hydraulic, pneumatic and/or other mechanical force; light; and/or sound.

Delivery device 100 can comprise one or more sensors, transducers, and/or other functional elements, functional element 199 shown and described herebelow.

Delivery device 100 includes various fluid pathways (e.g. lumens and other fluid pathways through which agent 200 is propelled), such as fluid pathways of connection layer 162 and other pathways of delivery device 100 (e.g. pump 161 and other pumping mechanism 160 components, reservoir 120, and TDA 130). These fluid pathways can include one or more coverings (e.g. films) and/or coatings (“coatings” herein) that can provide numerous advantages, such as is described herein. In some embodiments, a thin film or other coating is included to provide a particular level of permeability, such as to increase or limit permeability to a particular substance such as oxygen and/or water. In some embodiments, a coating is included to provide compatibility with agent 200 (e.g. to cover a surface that is not compatible with agent 200). In some embodiments, a coating is included selected from the group consisting of: antimicrobial; antibacterial; antifungal; anti-inflammatory; hydrophilic; hydrophobic; water-resistant; conductive; and combinations of these.

In some embodiments, system 10 comprises one or more agents for delivery by delivery device 100, agent 200 shown. Agent 200 can comprise one or more agents, such as one, two, or more agents selected from the group consisting of: an agent as defined herein; insulin; glucagon, glucose; dextrose; amylin; ghrelin; estrogen; progesterone; prolactin; testosterone; serotonin; cortisol; triiodothyronine; thyroxine; adrenaline; somatotropin; granulocyte-colony stimulating factor (GCSF); hepatocyte growth factor (HGF); and analogs and combinations of these.

In some embodiments, agent 200 comprises insulin with a concentration of at least 100 Units/mL, such as at least 200, 220, 300, and/or 500 Units/mL. In some embodiments, agent 200 comprises a concentrated ultra-rapid insulin (CURI). In some embodiments, agent 200 comprises an insulin analog, such as is described in U.S. Pat. No. 9,725,493.

In some embodiments, agent 200 comprises a first agent 200a and a second agent 200b, such as when delivery device 100 comprises two reservoirs 120 (e.g. as described in reference to FIG. 2A and/or 2B herein), and/or when system 10 comprises two delivery devices 100. In some embodiments, first agent 200a comprises insulin and second agent 200b comprises glucagon and/or glucose.

Agent 200 can comprise a first agent 200a and a second agent 200b that can be mixed together without deleterious effect, such as when first agent 200a and second agent 200b are both stored in a single reservoir 120. Alternatively, mixing of first agent 200a and second agent 200b may need to be avoided, and first agent 200a and second agent 200b can be stored in separate reservoirs 120 as described herein (e.g. and pumped by separate pumping mechanisms 160 and/or delivered to the patient by different TDAs 130).

In some embodiments, system 10 comprises one or more devices for communicating with delivery device 100, communication device 300 shown. Communication device 300 can comprise one or more sensors, transducers, and/or other functional elements, functional element 399 shown and described herebelow.

Communication device 300 can comprise a device selected from the group consisting of: a smart phone or other cell phone (e.g. the patient's cell phone); tablet; laptop computer; computer; personal data assistant; smart watch (such as an Apple Watch); special purpose communication device; and combinations thereof.

In some embodiments, communication device 300 comprises two or more communications devices, such as a first communication device 300a comprising a cell phone and a second communication device 300b comprising a laptop computer, or a device 300a comprising a cell phone and a device 300b comprising a smart watch. In these embodiments, delivery device 100 can communicate with (e.g. transfer data with) both device 300a and device 300b. For example, data provided by communication device 300a to delivery device 100 can be confirmed as being the same data (e.g. no missing data, two data sets that are exactly the same) provided to device 100 by communication device 300b (e.g. a redundancy check of data provided by either communication device 300). Alternatively or additionally, data provided by delivery device 100 to first communication device 300a can be confirmed as being the same data provided by device 100 to second communication device 300b.

In some embodiments, system 10 comprises one or more devices for filling device 100 with agent 200, fill device 400 shown. Fill device 400 can comprise one or more sensors, transducers, and/or other functional elements, functional element 499 shown and described herebelow.

In some embodiments, fill device 400 is configured to provide fluid to delivery device 100 at a flow rate of at least 5 mL/min, 10 mL/min, 20 mL/min, 60 mL/min, or 120 mL/min.

In some embodiments, delivery device 100 is provided to the patient already filled with agent 200, such that fill device 400 is used by the manufacturer of delivery device 100 but not needed by the patient using delivery device 100. For example, reservoir 120 can be filled using fill device 400 prior to its assembly within housing 110. The internal portions of reservoir 120 can be placed in a vacuum to assist in the filling of reservoir 120 with agent 200.

In some embodiments, system 10 comprises one or more devices for determining a physiologic parameter of the patient, sensing device 500 shown. Sensing device 500 can comprise one or more sensors, transducers, and/or other functional elements, functional element 599 shown and described herebelow.

Sensing device 500 can be configured to measure a patient physiologic parameter and/or a patient environment parameter (e.g. a measure of the temperature, pressure, humidity, and/or other parameter of the room or other environment in which the patient is currently present).

Sensing device 500 can be configured to measure a glucose level of the patient (e.g. blood glucose level), such as a measurement that is performed intermittently and/or continuously. Sensing device 500 can be configured to measure a patient physiologic parameter selected from the group consisting of: glucose level; blood glucose level; a blood gas parameter; blood pressure; heart rate; an electrocardiogram (EKG); an electroencephalogram (EEG); patient galvanic skin resistance (GSR) and/or electrodermal activity (EDA); a respiration parameter; pH; a hormone level; and combinations of these. In some embodiments, sensing device 500 is configured to measure a parameter of the environment of delivery device 100 and/or the patient, such as a parameter selected from the group consisting of: ambient temperature; ambient pressure; ambient humidity; and combinations of these.

In some embodiments, sensing device 500 is integrated into device 100, such as when sensing assembly 170 comprises sensing device 500.

In some embodiments, sensing device 500 comprises two or more sensing devices, such as a first sensing device 500a and a second sensing device 500b. In these embodiments, data provided by both sensing devices 500a and 500b (e.g. similar and/or dissimilar patient physiologic and/or patient environment data) can be used by algorithm 141 to adjust and/or otherwise control delivery of agent 200 to the patient.

In some embodiments, system 10 comprises at least one sensing device 500, and delivery device 100 comprises at least one sensing assembly 170. In these embodiments, data provided by both sensing devices 500 and sensing assembly 170 (e.g. similar and/or dissimilar patient physiologic and/or patient environment data) can be used by algorithm 141 to adjust and/or otherwise control delivery of agent 200 to the patient.

In some embodiments, system 10 comprises one or more devices for inserting a transcutaneous cannula or other component of delivery device 100 through the patient's skin, insertion device 600 shown. Insertion device 600 can comprise one or more controls (e.g. buttons), such as control 601 shown. Insertion device 600 can comprise one or more sensors, transducers, and/or other functional elements, functional element 699 shown and described herebelow.

Insertion device 600 can be configured to insert cannula 131 through the patient's skin. In some embodiments, insertion device 600 inserts both needle 132 and a surrounding cannula 131 through the patient's skin. In these embodiments, soon (e.g. immediately) after insertion of needle 132 and cannula 131, insertion device 600 can retract the needle 132 within cannula 131 such that needle 132 no longer passes through the patient's skin. In some embodiments, insertion device 600 automatically retracts needle 132 (e.g. after cannula 131 insertion without additional operator action). In some embodiments, insertion device 600 removes needle 132 from delivery device 100 after the insertion step.

Insertion device 600 can be configured to insert cannula 171 through the patient's skin. In some embodiments, insertion device 600 inserts both a needle 172 and a surrounding cannula 171 through the patient's skin. In these embodiments, soon (e.g. immediately) after insertion of needle 172 and cannula 171, insertion device 600 can retract the needle 172 within cannula 171 such that needle 172 no longer passes through the patient's skin. In some embodiments, insertion device 600 removes needle 172 from delivery device 100 after the insertion step.

In some embodiments, insertion device 600 is configured to insert both cannula 131 and cannula 171 through the patient's skin (e.g. by the same or similar insertion mechanisms). In these embodiments, insertion device 600 can be configured to insert cannula 131 and cannula 171 simultaneously or sequentially.

In some embodiments, the distance through the skin that insertion device 600 inserts cannula 131 and/or 171 (the “insertion distance”) is adjustable.

In some embodiments, the insertion distance for cannula 131 and the insertion distance for cannula 171 are different.

Insertion device 600 can comprise one or more scotch yoke mechanisms for inserting cannula 131, needle 132, cannula 171, and/or needle 172. The one or more scotch yoke mechanisms can be further configured to retract needle 132 and/or 172. In some embodiments, insertion device 600 comprises needle 132 and/or 172. Needle 132 and/or 172 can comprise a bevel that is oriented away from the skin of the patient during insertion. Needle 132 and/or 172 can comprise a five-edge bevel.

Cannula 131 and/or cannula 171 (e.g. including an inserted needle 132 and/or needle 172, respectively) can be configured to be inserted (e.g. inserted using insertion device 600) at an angle between 30° and 60° relative to the patient's skin.

In some embodiments, system 10 comprises one or more tools, tool 700 shown.

Tool 700 can comprise one or more sensors, transducers, and/or other functional elements, functional element 799 shown and described herebelow.

In some embodiments, tool 700 comprises a charging tool 700a comprising a charging device configured to charge (e.g. wirelessly recharge) power supply 150 of delivery device 100.

In some embodiments, tool 700 comprises a forming tool 700b comprising a film forming tool configured to produce reservoir 120, such as is described in reference to FIGS. 4A-E.

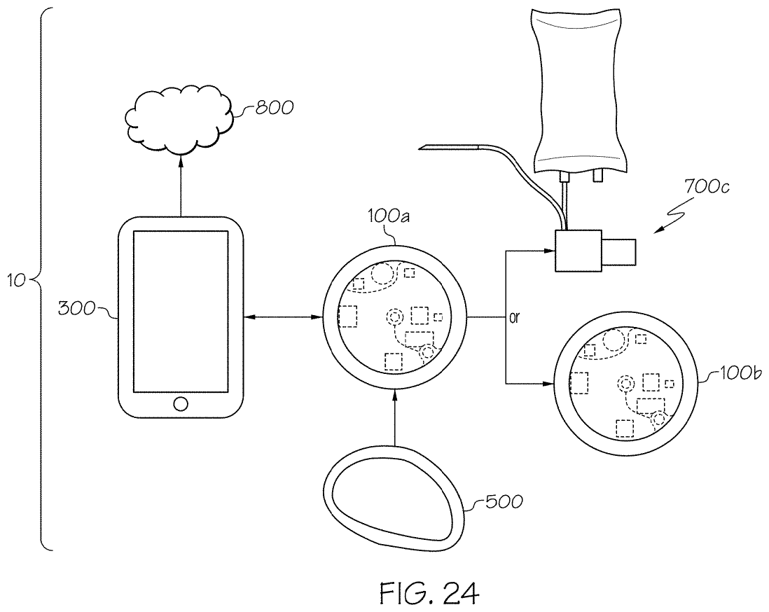

In some embodiments, tool 700 comprises a fluid delivery pump 700c configured to deliver one or more fluids to the patient, such as is described in reference to FIG. 24.

In some embodiments, system 10 comprises one or more computer networks, network 800 shown. Network 800 can comprise a network of one or more computers (e.g. one or more server computers), such as one or more computers controlled by a manufacturer of system 10. Network 800 can comprise the Internet, also referred to as “the cloud”. Network 800 can receive information from one or more components of system 10, such as delivery device 100, communication device 300, fill device 400, sensing device 500, insertion device 600, and/or tool 700. In some embodiments, network 800 receives information from delivery device 100, fill device 400, sensing device 500, insertion device 600, and/or tool 700 via communication device 300. In other words, the information is first transferred to communication device 300 from the associated system 10 component, and then transferred to network 800 by communication device 300. Data can be transmitted to a caregiver (e.g. a doctor or nurse) of the patient, and/or a third-party payor of the patient's therapy.

Network 800 can comprise one or more sensors, transducers, and/or other functional elements, functional element 899 shown and described herebelow.

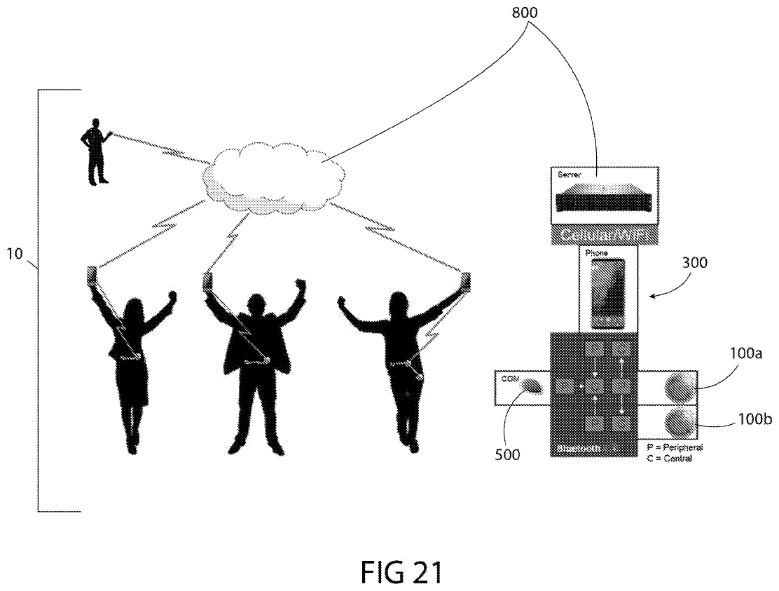

In some embodiments, network 800 is configured to analyze information received from a patient using system 10. In some embodiments, network 800 is configured to analyze information received from multiple patients using system 10. In some embodiments, network 800 is configured to anonymize the data received from a patient, for example to protect the privacy of the patient. For example, network 800 can be configured as described in reference to FIG. 21.

In some embodiments, system 10 comprises one or more sensors, transducers, and/or other functional elements, such as functional elements 99, 199 (of device 100), 399 (of device 300), 499 (of device 400), 599 (of device 500), 699 (of device 600), 799 (of tool 700), and/or 899 (of network 800), each as shown.

In some embodiments, a functional element 199 of delivery device 100 comprises a pressure sensor constructed and arranged to measure pressure of fluid proximate reservoir 120 (e.g. fluid within or near reservoir 120). In these embodiments, the functional element 199 can comprise a MEMS pressure sensor (e.g. Acuity 3012-030 pressure sensor and/or Acuity AC3031 pressure sensor), such as a pressure sensor with a parameter selected from the group consisting of: a range of 200 kPa to −200 kPa; a sensitivity of 1 Pa; a maximum range of 100 kPa; a size of 1.8 mm by 1.6 mm by 0.4 mm; a power of 5 mW; a range of 0 kPa to 5.3 kPa; a sensitivity of 8 Pa; a maximum pressure of 25 kPa; a size of 1.6 mm by 1.6 mm by 0.5 mm; a power of 7.1 mW; and combinations of these. This functional element 199 can further comprise a temperature sensor. This functional element 199 can be controlled by electronics 142 including signal conditioning circuitry (e.g. IDT ZSC31014) that can interface with (e.g. read, calibrate, and/or compensate output) functional element 199.

In some embodiments, a functional element 199 of delivery device 100 comprises a pressure sensor constructed and arranged to measure pressure of fluid proximate cannula 131 (e.g. fluid within or near cannula 131). In these embodiments, the functional element 199 can comprise a MEMS pressure sensor (e.g. Acuity AC3031 and/or Acuity 3012-030), such as a pressure sensor with a parameter selected from the group consisting of: a range of 0 kPa to 5.3 kPa; a sensitivity of 8 Pa; a maximum pressure of 25 kPa; a size of 1.6 mm by 1.6 mm by 0.5 mm; a power of 7.1 mW; a range of 200 kPa to −200 kPa; a sensitivity of 1 Pa; a maximum range of 100 kPa; a size of 1.8 mm by 1.6 mm by 0.4 mm; a power of 5 mW; and combinations of these. This functional element 199 can further comprise a temperature sensor. This functional element 199 can be controlled by electronics 142 including signal conditioning circuitry (e.g. IDT ZSC31014) that can interface with (e.g. read, calibrate, and/or compensate output) functional element 199.

In some embodiments, a functional element 199 of delivery device 100 comprises a sensor configured to measure a parameter within delivery device 100, such as a parameter associated with agent 200 maintained within delivery device 100.

In some embodiments, a functional element 199 of delivery device 100 comprises a sensor configured to measure a physiologic parameter of the patient, such as a physiologic parameter selected from the group consisting of: glucose level; blood glucose level; a blood gas parameter; blood pressure; heart rate; an electrocardiogram (EKG); an electroencephalogram (EEG); a respiration parameter; pH; a hormone level; and combinations of these.

In some embodiments, a functional element 199 of delivery device 100 comprises a sensor configured to detect a bubble, such as to detect a bubble present in: reservoir 120; pump 161, connection layer 162, and/or other portion of pumping mechanism 160; cannula 131 and/or other portion of TDA 130; and combinations of these.

In some embodiments, a functional element 199 of delivery device 100 comprises a sensor configured to detect a particle, such as to detect an undesired particle present in: reservoir 120; pump 161, connection layer layer 162, and/or other portion of pumping mechanism 160; cannula 131 and/or other portion of TDA 130; and combinations of these. For example, functional element 199 can comprise an LED-based particle scattering detector.

In some embodiments, a functional element 199 of delivery device 100 comprises a pressure sensor configured to measure a pressure within delivery device 100, such as to measure a pressure present in: reservoir 120; pump 161, connection layer layer 162, and/or other portion of pumping mechanism 160; cannula 131 and/or other portion of TDA 130; and combinations of these. For example, functional element 199 can comprise a pressure sensor configured to measure pressure of fluid in reservoir 120, such as a pressure sensor with a range of 200 kPa to −200 kPa, and/or with a sensitivity of 1 Pa. Alternatively or additionally, functional element 199 can comprise a pressure sensor configured to measure a pressure of fluid in TDA 130, such as a pressure sensor with a range of 0 kPa to 5.3 kPa, and/or with a sensitivity of 8 Pa.

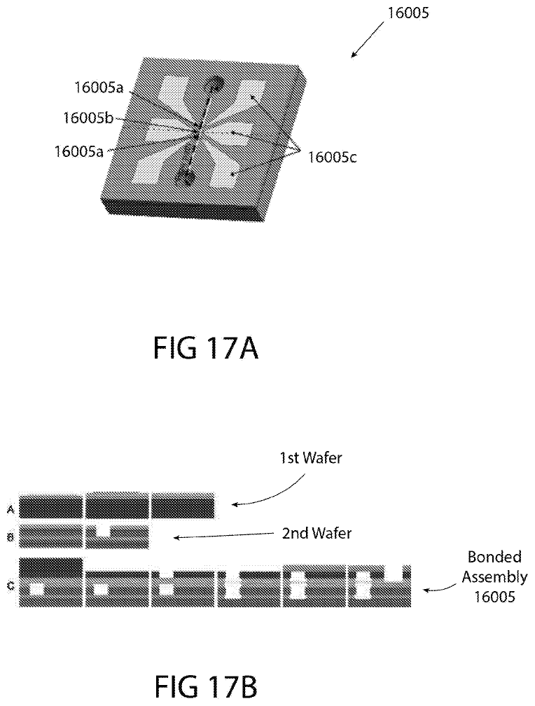

In some embodiments, a functional element 199 of delivery device 100 comprises a flow sensor configured to measure flow of fluid within delivery device 100, such as to measure a fluid flow present in: reservoir 120; pump 161, connection layer layer 162, and/or other portion of pumping mechanism 160; cannula 131 and/or other portion of TDA 130; and combinations of these. For example, functional element 199 can comprise an anemometer as described in reference to FIG. 17 herein.

In some embodiments, functional element 99 and/or 199 comprises a transducer configured to control the environment in which agent 200 is present, such as an internal portion of delivery device 100, or a container in which agent 200 is stored. For example, functional element 99 and/or 199 can comprise a transducer configured to cool agent 200 (e.g. a Peltier cooling element) and/or a transducer configured to warm agent 200. Alternatively or additionally, functional element 199 can comprise a transducer configured to agitate or otherwise cause motion within agent 200 (e.g. an eccentric motor assembly configured to vibrate reservoir 120).

In some embodiments, functional element 99, 199, 399, 499, 599, 699, 799, and/or 899 comprises a communication element, such as a Bluetooth or other wireless communication element. In some embodiments, the associated functional element is configured to provide a secure wireless link (e.g., using Bluetooth 4.1 or later) to BATDOC, Medhub, ACCS, and/or some other military or civilian medical information hub or system. For example, a communication-based functional element of one component of system 10 can transfer and/or receive information from a communication-based functional element of another component of system 10. In some embodiments, device 100 or another component of system 10 transfers data to and/or from communication device 300. In these embodiments, communication device 300 can transfer data to and/or from network 800, such as when network 800 is configured to analyze data produced by one or more patients using system 10 (e.g. as described in reference to FIG. 21).

In some embodiments, functional element 99, 199, 399, 499, 599, 699, 799, and/or 899 comprises an inertial measurement unit (IMU) and/or other motion sensor, such as a motion sensor configured to measure motion of the patient (e.g. for algorithm 141 to assess patient activity) and/or a motion sensor configured to measure motion of the associated component of system 10 (e.g. to determine if the component has been dropped).

In some embodiments, functional element 99, 199, 399, 499, 599, 699, 799, and/or 899 comprises a transducer, such as a transducer configured to provide an audible, visible, and/or tactile alarm that can be activated when device 100 and/or another component of system 10 enters an alarm state.

Referring now to FIG. 2A, a schematic view of a delivery system including a dual-agent delivery device is illustrated, consistent with the present inventive concepts. System 10 of FIG. 2A includes delivery device 100′ which can be configured to store (e.g. independently store) and deliver (e.g. independently deliver) two different agents 200, such as first agent 200a and second agent 200b shown. System 10 of FIG. 2A and its components can be of similar construction and arrangement to system 10 and its components of FIG. 1. Delivery device 100′ of FIG. 2A can include tubes 101, valves 102, bubble capture element 105, housing 110, control module 140, power supply 150, and/or functional element 199, each as described herein. Device 100′ can comprise two separate reservoirs 200, such as reservoirs 120a and 120b shown. In some embodiments, device 100′ comprises a single pumping mechanism, pumping mechanism 160, which can be configured to propel fluid received from both reservoir 120a and 120b. Alternatively, device 100′ can comprise two separate pumping mechanisms, mechanisms 160a and 160b shown, where mechanism 160a propels fluid received from reservoir 120a and mechanism 160b propels fluid received from reservoir 120b. Device 100′ can comprise a single TDA 130 which can be configured to deliver fluid stored in both reservoir 120a and 120b (e.g. as propelled by a single or multiple pumping mechanisms 160). Alternatively, device 100′ can comprise two separate transcutaneous delivery assemblies, such as TDA 130a and TDA 130b shown.

Each reservoir 120 of delivery device 100′ can comprise a fill port 121 and/or a drain port 122, each as shown and described herein.

Each TDA 130 of delivery device 100′ can comprise a cannula 131 and/or a needle 132 (e.g. a needle inserted within cannula 131), each as shown and described herein. In some embodiments, delivery device 100′ can comprise one or more integrated insertion assemblies, such as the two separate insertion assemblies 135a and 135b shown (e.g. for inserting cannula 131a and 131b, respectively, through the skin of the patient). Alternatively or additionally, system 10 of FIG. 2A can comprise a separate insertion device, such as insertion device 600 described in reference to FIG. 1 herein.

Each pumping mechanism 160 of delivery device 100′ can comprise a pump 161 and/or a connection layer 162, each as shown and described herein.

Referring now to FIG. 2B, a schematic view of a system for delivering one or more agents to a patient is illustrated, consistent with the present inventive concepts. System 10 includes an agent delivery device, device 100″ shown. Device 100″ comprises a housing 110, a reservoir 120A, a fluidics assembly 160A, a transcutaneous delivery assembly (“TDA” herein) 130A, a control module 140, and a power supply 150, each as shown.

Delivery device 100″ can comprise a mass of less than 20 g, such as less than 15, 12, 10, 9, or 8 g. Delivery device 100″ can comprise a dimension (e.g. a major axis) that does not exceed 55 mm, such as when device 100″ does not comprise a dimension that exceeds 45, 40, 38, 36, or 33 mm. Delivery device 100″ can comprise a height that does not exceed 10 mm, such as a height that does not exceed 8, 7.5, or 6.5 mm.

Delivery device 100″ can be configured to deliver insulin and/or an insulin analog (e.g. agent 200A) to the patient for at least three days, such as at least five or at least seven days.

Delivery device 100″ can be configured to be usable for at least 90 days, 120 days, 180 days, 270 days and/or 365 days after filing, such as when device 100″ is stored at a temperature no greater than 30° C.

Delivery device 100″ can further comprise a first portion 1025 that is configured to be rotated relative to one or more other portions of device 100″ (e.g. rotate relative to the remaining portions of device 100″). In some embodiments, first portion 1025 comprises reservoir 120A. Rotation of first portion 1025 can be configured to fluidly connect reservoir 120A to a pumping mechanism, fluidics assembly 160A. In some embodiments, the fluidic connection occurs by flow port 1605 of fluidics assembly 160A fluidly communicating with an access port, port 1204 shown, of reservoir 120A. In other embodiments, the connection occurs by flow port 1605 (e.g. a needle) of fluidics assembly 160A piercing a fill port, port 1201 shown (e.g. a septum) of reservoir 120A. Rotation of first portion 1025 can be configured to activate (e.g. enable power or otherwise turn on) device 100″ and/or to initiate a priming protocol of fluidics assembly 160A. Rotation of first portion 1025 can be activated by an insertion device, such as insertion device 600 described herein.

Delivery device 100″ can comprise a second portion 1020 that is configured to be rotated relative to one or more other portions of device 100″ (e.g. rotate relative to the remaining portions of device 100″). Second portion 1020 can include reservoir 120A and fluidic assembly 160A (e.g. second portion 1020 can include first portion 1025). Rotation of second portion 1020 (e.g. subsequent to the rotation of first portion 1025 described hereabove) can cause fluidic assembly 160A to fluidly connect with TDA 130A, such as to create a fluidic pathway from reservoir 120A to TDA 130A. For example, rotation of second portion 1020 can cause an output port (e.g. an output port 1605 including a needle) of fluidics assembly 160A to fluidly connect with a proximal portion of TDA 130A (e.g. the needle piercing a septum, septum 1301, of TDA 130A). Once rotated, TDA 130A can be primed. Rotation of second portion 1020 can be activated by an insertion device, such as insertion device 600 described herein.

Delivery device 100″ can further comprise a pad, pad 1001 shown, which can be configured to collect froth, for example during the process of priming the fluidics assembly 160A. In some embodiments, pad 1001 is positioned proximate TDA 130A and/or fluidics assembly 160A, for example proximate to an output port 1605.

Delivery device 100″ and/or another component of system 10 can further comprise one or more sensors, sensor 170 shown. Sensor 170 can comprise at least one environmental sensor configured to monitor a parameter selected from the group consisting of: pressure; temperature; humidity; other environmental factors; and combinations of these. Sensor 170 can comprise at least one sensor configured to monitor one or more parameters of an agent, such as agent 200A. In some embodiments, an agent-based sensor 170 is configured to detect bubbles, particles, contaminants, and/or degradation products within agent 200A. In some embodiments, an agent-based sensor 170 is configured to scatter light. Sensor 170 can comprise at least one sensor configured to monitor one or more patient parameters. In some embodiments, sensor 170 is configured to monitor a patient parameter selected from the group consisting of: blood glucose; motion; activity; heart rate; EKG; respiration; EEG; a tissue gas parameter; a hormone level; pH; blood oxygen saturation; a photospectrographic tissue state; electrodermal activity (EDA); and combinations of these. Sensor 170 can comprise one or more sensors that are positioned in contact with the patient's dermis, epidermis, and/or subcutis. Sensor 170 can comprise one or more sensors that are configured to be inserted into the patient's subcutaneous tissue. For example, patient sensor 170 can comprise an insertable glucose sensing device that includes a skin-penetration portion.

Delivery device 100″ can comprise an assembly, transceiver 1010, which can be configured to send and/or receive wireless transmissions to one or more other components of system 10. Delivery device 100″ can be configured to communicate (e.g. continuously and/or intermittently) with one or more external devices (e.g. one or more other devices of system 10 as described herein). Delivery device 100″, via transceiver 1010, can communicate with an external device via a wireless protocol, such as a Bluetooth connection, such as Bluetooth 4.1, 4.2, or greater or their equivalent.

Delivery device 100″ can further comprise at least one skin attachment element, attachment element 1002. Attachment element 1002 can be constructed and arranged similar to skin attachment assembly 111 as described herein. Attachment element 1002 can comprise an adhesive patch. Attachment element 1002 can be configured to provide at least 3 days of attachment to the patient, such as at least 5, 7, or 10 days of attachment to the patient. Attachment element 1002 can be configured for easy, comfortable, removal from the patient.

Delivery device 100″ and/or system 10 can further include an assembly for delivering feedback to a user (e.g. the patient), feedback module 1030. Feedback module 1030 can comprise a haptic device 1031 configured to provide information via haptic feedback to the patient, such as alarm or other alert information (“alert information” herein). Feedback module 1030 can comprise an auditory device 1032 configured to provide information via sound to the patient, such as alert information. In some embodiments, auditory device 1032 comprises a piezo actuator. Feedback module 1030 can comprise a visual device 1033 configured to provide information via visual feedback to the patient, such as alert information. In some embodiments, visual device 1033 comprises a light, such as an LED light. Alert information can include information relate to an alarm condition, information related to an action to be performed by the patient, and/or other types of alerts (e.g. requiring or suggesting action by the patient or other operator of system 10).

Delivery device 100″ can further comprise fluid resistance element 1003, which can be configured to protect electronics and/or other components within device 100″ from damage from water and/or another fluid (e.g. by reducing fluid ingress into housing 110 and/or preventing fluid already inside housing 110 from contacting components, such as components of the control module 140). In some embodiments, fluid resistance element 1003 comprises a coating, such as a polymer coating.

Delivery device 100″ can further comprise a second reservoir, reservoir 120B shown. Second reservoir 120B can be constructed and arranged similar to reservoir 120A as described herein, such that second reservoir 120B comprises a second fluidics assembly, assembly 160B shown, and/or a second TDA, TDA 130B shown. Reservoir 120B is constructed and arranged to house a second agent, such as agent 200B. In some embodiments, agent 200B comprises glucagon and/or a glucagon analog.

Housing 110 can comprise one, two, or more openings configured to: provide drainage; provide barometric pressure compensation for reservoir 120A; allow filling of reservoir 120A; enable attachment to, and actuation by, an insertion device 600; allow insertion of cannula 1302 (described herebelow) by insertion device 600; allow insertion of a glucose sensor 170 by insertion device 600; and combinations of these.

Housing 110 can further comprise a transparent window, window 1101, which can be constructed and arranged to align with a transparent window of reservoir 120A, window 1205 shown.

Reservoir 120A can comprise a rigid, solid base, base 1203. Base 1203 can comprise a permeation-resistant polymer that is compatible with agent 200A. In some embodiments, base 1203 comprises cyclic olefin copolymer (COC). Reservoir 120A can further comprise a flexible film cover, cover 1206 shown. Cover 1206 can comprise a laminate that is resistant to permeation, such as a laminate resistant to the permeation of water and/or oxygen. In some embodiments, cover 1206 comprises at least one of COC or Polychlorotrifluoroethylene (PCTFE). Cover 1206 can be constructed and arranged as a “rolling-sock structure”. Cover 1206 can comprise a structure that resists stretching in at least one direction, and/or a structure that includes at least one portion that stretches and at least one portion that does not stretch.

Reservoir 120A is constructed and arranged to house first agent 200A. In some embodiments, device 100″ is provided to the patient (e.g. shipped or otherwise delivered to the patient) pre-filled with first agent 200A. Reservoir 120A can house an amount of insulin or formulation of an insulin analog (either or both “insulin” herein) equal to at least 300 Units of insulin, such as at least 400, 500, 600, or 800 Units of insulin. Reservoir 120A can house at least a three-day supply of agent 200A (e.g. insulin), such as at least a four, five, seven, or fifteen-day supply of agent 200A. Agent 200A can be configured to resist degradation. For example, agent 200A (e.g. insulin) can be configured to retain at least 95% potency when stored in reservoir 120A for at least one month at a temperature of no more than 30° C., such as when stored in reservoir 120A for at least three, four, five, six, eight, or twelve months at a temperature of no more than 30° C. Agent 200A can comprise an insulin analog with a concentration of at least 100 Units/mL, such as a concentration of at least 200, 220, 300, and/or 500 Units/mL.

Agent 200A can comprise an agent selected from the group consisting of: insulin; one or more insulin analogs; glucagon; glucose; dextrose; amylin; ghrelin; estrogen; progesterone; prolactin; testosterone; serotonin; cortisol; triiodothyronine; thyroxine; adrenaline; somatotropin; GCSF; HGF; GLP-1 receptor agonists; DPP-4 inhibitors; meglitinides; SGLT-2 inhibitors; sulfonylureas; thiazolidinediones; analogs of these; and combinations of these.

Reservoir 120A can comprise a fill volume of less than 2.4 mL, such as a fill volume of less than 2.0, 1.5, 1.3, 1.2, 0.8, 0.2, or 0.1 mL.

Reservoir 120A can further comprise a fill port 1201 that can include a septum or plug. Fill port 1201 can be constructed and arranged similar to fill port 121 as described herein.

Reservoir 120A can further comprise an access port 1204 that can include a septum or plug. In some embodiments, access port 1204 is positioned proximal to fill port 1201.

Reservoir 120A can further comprise a bubble trap, bubble trap 1202 shown. Bubble trap 1202 can be constructed and arranged similar to bubble capture element 105 as described herein. In some embodiments, bubble trap 1202 is positioned proximal to access port 1204.

Reservoir 120A can further comprise transparent window 1205.

Fluidics assembly 160A can comprise a pumping mechanism selected from the group consisting of: a piston pump; a syringe pump; a peristaltic pump; a micro-electromechanical system (MEMS) pump; a displacement pump; and combinations of these.

Fluidics assembly 160A can comprise one, two, or more micro-electromechanical system (MEMS) components, components 1600 shown. MEMS components 1600 can be manufactured using semiconductor fabrication techniques.

MEMS components 1600 can comprise a pumping actuator, actuator 1601 shown. Actuator 1601 can comprise one or more actuator valves. Actuator 1601 can comprise one or more drive elements. At least one drive element of actuator 1601 can comprise a piezo actuator. At least one drive element can comprise a silicon diaphragm actuated via micro-machined PZT film. The one or more drive elements can comprise at least one dimension that is less than 2.2 mm, such as at least one dimension that is less than 2.1, 2.0, 1.5, 0.8, 0.6, or 0.1 mm. For example, at least one drive element can comprise an element with dimensions of approximately 2.1×2.2×0.06 mm. Actuator 1601 can comprise at least one chamber configured to be actuated to take in and/or expel contents within actuator 1601. Actuator 1601 can comprise at least two, three, four, or five chambers. Each chamber can comprise a volume of at least 1 nL, such as a volume of at least 5, 10, 15, 20, 30, or 50 nL. Two or more chambers of actuator 1601 can be connected via at least one microfluidic channel. Actuator 1601 can comprise a stroke volume of less than 50 nL, such as a stroke volume of less than 25, 15, 10, 5, 2, or 1 nL. Actuator 1601 can be configured to produce an output pressure of at least 10 kPa, such as a pressure of at least 20, 30, 60, 80, 100, or 200 kPa. Actuator 1601 can comprise one or more agent-contacting surfaces that comprise a coating selected from the group consisting of: antimicrobial; antibacterial; antifungal; anti-inflammatory; hydrophilic; hydrophobic; water-resistant; conductive; and combinations of these. In some embodiments, the coating comprises a thin film applied to the one or more agent-contacting surfaces of actuator 1601.

MEMS components 1600 can comprise one or more sensors, sensor 1602. At least one sensor 1602 can be positioned proximate reservoir 120A. At least one sensor 1602 can be positioned proximate TDA 130A. In some embodiments, at least one sensor 1602 comprises a pressure sensor. Sensor 1602 can comprise one or more pressure sensors configured to operate in a range of 0, −10, −50, −100, −200, −300, −500 kPa to 10, 50, 100, 200, 300, 500 kPa. Sensor 1602 can comprise a pressure sensor with a sensitivity of −1, 1, 2, 4, 8, or 10 kPa. In some embodiments, at least one sensor 1602 comprises a fluid flow sensor. Sensor 1602 can comprise one or more fluid flow sensors selected from the group consisting of: a differential pressure sensor; a positive displacement flow meter; a velocity flow meter; a mass flow meter; an open-channel flow meter; and combinations of these. Sensor 1602 can comprise a fluid flow sensor that includes a hot wire anemometer. In some embodiments, a hot wire anemometer of sensor 1602 comprises platinum. In some embodiments, the hot wire anemometer of a sensor 1602 comprises one heated wire and two unheated wires.

MEMS components 1600 can comprise one or more fluid flow control valves, valve 1607 shown. Valve 1607 can comprise one or more active valves, and/or one or more passive valves. Valve 1607 can comprise one or more valves that are configured to block undesired fluid flow at up to a pressure of at least 1000 kPa, such as a pressure up to at least 2000, 5000, or 10,000 kPa. In some embodiments, valve 1607 comprises one or more valves that are configured to prevent backflow of fluid (e.g. agent 200A), such as to passively prevent backflow. In some embodiments, valve 1607 comprises one or more valves that are configured to prevent excess forward flow of fluid (e.g. agent 200A), such as to passively prevent excess forward flow. In some embodiments, valve 1607 comprises two or more valves (e.g. including at least one active valve) that are included in a redundant arrangement (e.g. a serial arrangement), such as to actively provide redundant safety (e.g. when a first valve fails, the second valve prevents occurrence of an undesired condition).

Fluidics assembly 160A can comprise various electronic components configured as an electronic control module, control electronics 1603 shown. Electronics 1603 can comprise a control algorithm configured to monitor and/or control various parameters of device 100″, such as temperature, pressure, flow rate, and/or fluid volume. In some embodiments, electronics 1603 causes device 100″ to enter an alert state (e.g. a warning or alarm state) if pressures, temperatures, and/or other sensed parameters of fluidics assembly 160A exceed a threshold. In some embodiments, electronics 1603 shuts down flow if pressure, temperature, and/or other sensed parameters of fluidics assembly 160A exceed a threshold.

Fluidics assembly 160A can comprise a micro-fluidics routing layer, layer 1604 shown. In some embodiments, MEMS components 1600 described herein are bonded to micro-fluidics layer 1604. Micro-fluidics layer 1604 can comprise one, two, or more microfluidic channels. Micro-fluidics layer 1604 can comprise one, two, or more fiducial marks. Fiducial marks can be included, such as to aid in positioning of one or more discrete components of MEMS components 1600. Micro-fluidics layer 1604 can comprise a needle-seating feature. Micro-fluidics layer 1604 can comprise a channel including a fileted corner. Micro-fluidics layer 1604 can be manufactured from COC and/or poly(methyl acrylate) (PMA). Micro-fluidics layer 1604 can comprise a hydrophilic coating (e.g. a coating applied to the microfluidic channels of layer 1604).

Fluidics assembly 160A can comprise one, two, or more flow ports, ports 1605 shown. Ports 1605 can comprise needles (e.g. steel needles) with a gauge selected from the group consisting of: 34; 32; 30; 28; 26; 24; and combinations of these. The needles of ports 1605 can extend at least 0.5, 1, 2, 3 mm from fluidics assembly 160A. In some embodiments, ports 1605 comprise non-coring needles or needles that are otherwise designed to fluidically and atraumatically engage with a septum without causing significant damage to the septum and providing a fluidic seal during engagement. In some embodiments, ports 1605 comprise needles bonded to micro-fluidics layer 1604. At least one flow port 1605 can be positioned proximal to reservoir 120A. At least one flow port 1605 can be positioned proximal to TDA 130A. At least two flow ports 1605 can be positioned on opposite ends of fluidics assembly 160A.

Fluidics assembly 160A can comprise an element configured to capture a gas bubble, bubble catcher 1606. Catcher 1606 can be constructed and arranged similar to bubble capture element 105 as described herein. Catcher 1606 can be positioned proximal to reservoir 120A (e.g. in a fluid channel at a location proximate reservoir 120A).

Fluidics assembly 160A can comprise a “system on a chip” or a “system in a package” arrangement, such as an arrangement where multiple components and control circuits are integrated (e.g. using semiconductor or other automated assembly equipment) in a single chip package. MEMS components 1600 can comprise one or more electronic components that are bonded (e.g. wirebonded) to a circuit board. Electronics 1603 can comprise one or more components that are bonded (e.g. wirebonded) to a circuit board. Micro-fluidics layer 1604 can be bonded to a circuit board. Fluidics assembly 160A can be assembled and/or sealed using semiconductor packaging techniques. The sealing can comprise a polymer housing and/or filling material. The filling material may comprise epoxy and/or fluorosilicone gel.

Fluidics assembly 160A can comprise a curved geometry (e.g. at least a portion of fluidics assembly 160A comprises a curved geometry). The curved geometry can comprise an annulus sector or segment.

Fluidics assembly 160A can be positioned proximate the periphery of the housing 110 of delivery device 100″.

Fluidics assembly 160A can be configured to produce an output pressure of greater than 10 kPa, such as an output pressure of greater than 30, 60, 100, 200, or 500 kPa.

Fluidics assembly 160A can produce an output pressure configured to deliver a bolus of agent 200A (e.g. a bolus delivered to subcutaneous tissue) of at least 25 nL within a one second time period, such as a bolus of at least 50, 100, or 200 nL within a one second time period.

TDA 130A can comprise a proximal portion comprising a septum 1301 and a distal portion comprising a skin-penetrating element, cannula 1302. Cannula 1302 can be configured to provide fluid access into: an artery; a vein; tissue; an organ; the interstitial space between organs or tissue planes; and combinations of these. The tissue can be subcutaneous adipose tissue. For example, cannula 1302 can be configured to access the intraperitoneal space and/or the tissue planes around the peritoneum. In some embodiments, at least a portion of cannula 1302 is placed to deliver fluid subcutaneously and/or into a blood vessel (e.g. an artery or a vein). Cannula 1302 can be configured to be inserted into and/or through an access assembly (e.g. a septum) of an implanted access port. The access port can include a catheter configured to deliver fluid (e.g. agent 200A as provided by device 100″) to an anatomical location selected from the group consisting of: artery; vein; tissue; organ; interstitial space between organs or tissue planes; intraperitoneal space and/or the tissue planes around the peritoneum; and combinations of these. Cannula 1302 can comprise one or more metal or polymeric materials. The polymeric material can be flexible, and/or it can include one or more flexible portions. Cannula 1302 can comprise an internal metal coil. Cannula 1302 can comprise multiple outlets (e.g. multiple holes exiting the wall of a shaft of cannula 1302). Cannula 1302 can comprise a shaft that is inserted through the skin via an internal needle. In some embodiments, cannula 1302 remains outside of the patient, such as when cannula 1302 is connected to a separate device that passes through the patient's skin (e.g. cannula 1302 is connected to an intravenous line, an arterial access device, a subcutaneously placed access port, an infusion set, and the like).

TDA 130A can be inserted and/or connected to device 100″ as TDA 130A is inserted into the patient. In some embodiments, TDA 130A is connected to device 100″ via an insertion device (not shown but similar to insertion device 600). In some embodiments, cannula 1302 of TDA 130A is inserted through the skin of the patient by an insertion device, such as insertion device 600 described herein. In some embodiments, all or a portion of insertion device 600 is integral to device 100″, such as when device 100″ includes insertion assembly 135 described herein in reference to FIG. 1.

Control module 140 can comprise a microprocessor or other microcontroller, microprocessor 1401 shown. Control module 140 can further comprise one, two, or more accessory electronics, electronics 1403 shown. Accessory electronics 1403 can comprise one or more components configured to provide communication between two or more components of system 10 (e.g. two or more components of fluidics assembly 160A). Accessory electronics 1403 can include one or more memory components configured to provide data storage. Accessory electronics 1403 can be configured to provide power regulation. Accessory electronics 1403 can be configured to provide communication with one or more components external to delivery device 100″ (e.g. one or more other components of system 10). Accessory electronics 1403 can comprise an electronics module comprising one or more of: a real time clock; actuator control circuits; and/or sensor control circuits.

Control module 140 can comprise one or more algorithms, algorithm 1402 shown. Algorithm 1402 can be configured similar to algorithm 141 as described herein. Algorithm 1402 can comprise one or more algorithms configured to control the delivery of one or more agents 200A. In some embodiments, algorithm 1402 alters the flow rate in response to agent 200A fluid flow information and/or agent 200A fluid pressure information (e.g. as monitored by sensor 170 or sensor 1602 described herein). Algorithm 1402 can be configured to determine the delivery rate of agent 200A based on: current and/or historical data from sensor 170 and/or other sensor of system 10; previous deliveries of agent 200A; the patient's previous response (e.g. physiologic response) to agent 200A; patient disclosed-plans, such as when the patient-disclosed plan relates to meal size, meal composition, exercise, work activities, stress-inducing activities, and/or other activities planned by the patient; parameters communicated to control module 140 via communication device 300, such as when these parameters alter how algorithm 1402 responds to other inputs and/or recognizes patterns in the other inputs or data algorithm 1402 can access; and combinations of these. Algorithm 1402 can comprise at least one algorithm that is configured as a learning algorithm, such as a machine learning algorithm. In some embodiments, algorithm 1402 is configured as a parameter-based algorithm such that the one or more parameters (e.g. flow parameters and/or responsiveness to changes in sensor data and/or weight placed on historical flow, pressure, and/or sensor data) are adjusted based on the patient's historical response (e.g. physiologic response) to receiving agent 200A delivered by one or more delivery devices 100″ (e.g. via data collected by one or more sensors 170 of system 10). In some embodiments, the parameters are received via the transceiver 1010. In some embodiments, the parameters are received from a communications device 300 and/or a computer network, network 800 (each described herein), for example during the start-up of a delivery device 100. In some embodiments, the parameters are sent via transceiver 1010 to a communication device 300, and/or to network 800 for storage. Algorithm 1402 can comprise at least one algorithm that is configured to bias the delivery of agent 200A to under deliver agent 200A (e.g. such as to bias away from potential over delivery of agent 200A).

Algorithm 1402 can comprise at least one algorithm that is configured to trigger one or more alerts (e.g. audible, visual, and/or tactile alerts) when one or more system 10 operational parameters (e.g. as measured by sensor 170) and/or patient parameters (e.g. as measured by sensor 170) exceed a pre-determined threshold.

Algorithm 1402 can comprise at least one algorithm that is configured to determine one or more of: which data is stored by system 10 (e.g. which one or more types of data are stored by system 10); the method of data (e.g. any data) storage used by system 10, and/or one or more locations of data (e.g. any data) stored by system 10.

Algorithm 1402 can comprise at least one algorithm that is configured to determine which data is communicated to other components of system 10, and/or how data (any data) is communicated by device 100″.

Power supply 150 can comprise a battery, capacitor, and/or other energy storage component. Power supply 150 can be rechargeable, such as a supply configured to be rechargeable via wireless transmissions of power. Power supply 150 can comprise a super capacitor. Power supply 150 can comprise a relatively flat geometry. In some embodiments, power supply 150 is positioned proximate reservoir 120A. In some embodiments, power supply 150 is positioned parallel to a main plane of reservoir 120A. Power supply 150 can comprise a power supply configured to provide (e.g. after a single charge) at least 50 joules, such as at least 100, 500, 1000, or 2000 joules of energy. Power supply 150 can be configured to provide power to device 100″ for at least three days, such as at least five, seven, eight, nine, or ten days.

System 10 can further comprise insertion device 600 shown, which can be configured to insert a portion of TDA 130A (e.g. cannula 1302) through the patient's skin (e.g. such that the end or other fluid delivery portion of cannula 1302 is positioned at a target location for the delivery of agent 200A), as described herein. In some embodiments, insertion device 600 is configured to insert one or more sensors, such as sensor 170, through the skin of a patient (e.g. to implant sensor 170 in subcutaneous tissue, subdermal space, and/or other internal body location). For example, the inserted sensor can comprise a blood glucose sensor that provides information to device 100″ for the delivery of insulin. In some embodiments, insertion device 600 is configured to insert cannula 1302 and sensor 170 through the patient's skin in a simultaneous and/or sequential arrangement.

Insertion device 600 can be configured to be removably attached to delivery device 100″. Insertion device 600 can be configured to be removably attached to TDA 130A and/or to a sensor 170 to be inserted. Insertion device 600 can comprise one, two, or more needles, such as a metal needle. For example, the needle can be initially positioned within cannula 1302 of TDA 130A prior to and during the insertion of cannula 1302 through the patient's skin. Insertion device 600 can be configured to contain TDA 130A and/or sensor 170 prior to activation. In some embodiments, insertion device 600 can be activated to transfer TDA 130A and/or sensor 170 to the delivery device 100″. The transference can be configured to occur contemporaneously with insertion of TDA 130A and/or the insertion of sensor 170 into the patient's skin.

Insertion device 600 can include insertion assembly 635, which can comprise a mechanism configured to advance cannula 1302 through the patient's skin. Insertion device 600 can include one or more controls, control 601 shown, configured to initiate the advancement of cannula 1302 through the skin of the patient by insertion assembly 635. In some embodiments, the distance of insertion of cannula 1302 is adjustable, such as an adjustment performed via a control 601 configured to adjust the insertion performed by assembly 635. In some embodiments, insertion device 600 includes a needle (not shown but such as needle 132 described herein) which advances into the tissue with cannula 1302 during insertion, and is withdrawn from the cannula 1302 (and from the patient) by device 600 and/or when device 600 is detached from TDA 130A. In some embodiments, the needle is automatically withdrawn from the patient consequent to activation of insertion device 600 by controls 601.

In some embodiments, sensor 170 includes a portion that is implanted in the patient, and a portion that extends through the patient's skin and operably attaches to electronics of delivery device 100″. In some embodiments, the extending portion of sensor 170 can be configured to operably attach to the electronics of delivery device 100″ in a procedure in which sensor 170 is implanted in the patient. In some embodiments, a seal, such as a liquid-resistant seal, can be created between the extending portion of sensor 170 and housing 110, such as a seal that is created during a procedure in which sensor 170 is implanted in the patient.

System 10 can further comprise a communication device. device 300 shown, which can be configured to communicate information (“information” or “data” herein) to and/or from the patient. In some embodiments, system 10 comprises a continuous glucose monitor, CGM 500. CGM 500 can comprise a device configured to transmit patient glucose data to delivery device 100″ and/or communication device 300. In some embodiments, communication device 300 communicates intermittently with delivery device 100″ and/or CGM 500. Communication device 300 can be configured to communicate information audibly, tactilely, and/or visually to and/or from the patient. Communicate device 300 can be configured to receive information from the patient, such as information comprising a patient-plan. Communication device 300 can be configured to receive data from delivery device 100″, such as via transceiver 1010. In some embodiments, communication device 300 receives data collected by sensor 170 (e.g. data sent from delivery device 100″ and/or directly from sensor 170). In some embodiments, communication device 300 receives data related to delivery of agent 200A from delivery device 100″. Communication device 300 can be configured to send and/or receive data to and/or from a computer network 800. In some embodiments, communication device 300 receives performance data for an algorithm 1402 via a computer network, network 800 shown. In some embodiments, communication device 300 is configured to receive messages for the patient via network 800.

Communication device 300 can comprise a device selected from the group consisting of: smart phone; cell phone; tablet; laptop computer; computer; personal data assistant; smart watch; special purpose communication device; and combinations of these. Communication device 300 can be configured to send and/or receive data.

System 10 can further comprise network 800 as described herein. Network 800 can be configured to receive data from delivery device 100″ (via transceiver 1010) and/or communication device 300. Network 800 can be configured to send data to communication device 300 and/or delivery device 100″. Network 800 can be configured to store data, such as data received from communication device 300 and/or delivery device 100″. Network 800 can be configured to transmit data to a third-party, such as a caregiver, a third-party payor, friend, family member, clinician, diabetes educator, and the like. In some embodiments, network 800 transmits telemetry data to the third-party. Network 800 can be configured to (e.g. via an algorithm of system 10) perform an analysis on the data received from one or more communication devices 800 and/or delivery devices 100″. In some embodiments, network 800 (e.g. via an algorithm of system 10) performs an analysis on data collected from multiple patients (e.g. multiple delivery devices 100″). In some embodiments, network 800 utilizes machine learning to perform the analysis. Network 800 can be configured to transmit data and/or analyses of data to a payor (e.g. insurance provider or other third-party payor).

System 10 can further comprise a fill device, device 400 configured to fill reservoir 120A. Fill device 400 can be configured to fill reservoir 120A at a rate of at least 1 mL/min, such as at least a rate of at least 5, 10, 20, and/or 100 mL/min. Fill device 400 can be configured to evacuate gases from reservoir 120A. Fill device 400 can be configured to insert a plug into fill port 1201 (e.g. after filling of reservoir 120A). In some embodiments, fill device 400 is configured to fill reservoir 120A while the reservoir is inside of delivery device 100″. For example, device 100″ can be constructed and arranged such that reservoir 120A is positioned inside housing 110 prior to filling. Device 100″ can also be attached to insertion device 600 prior to filling. In other embodiments, fill device 400 is configured to fill reservoir 120A while the reservoir is outside of device 100″ (e.g. inside insertion device 600), or device 400 can be configured to fill reservoir 120A prior to the assembly of device 100″ (e.g. prior to the assembly of reservoir 120A with other components of device 100″).

System 10 can further comprise one, two, or more additional delivery devices, such as delivery devices 100′″ and 100″″ shown. Delivery devices 100′″ and/or 100′″ can be of similar construction and arrangement as delivery device 100, delivery device 100′, and/or delivery device 100″ described herein. Delivery devices 100′″ and/or 100′″ can be configured to receive instructions from delivery device 100″. In some embodiments, delivery devices 100′″ and/or 100″″ are controlled via delivery device 100″. Delivery devices 100′″ and/or 100″″ can be configured to receive data from and/or transmit data back to delivery device 100″. Alternatively or additionally, delivery devices 100′″ and/or 100′″ can be configured to operate independently of delivery device 100″. In some embodiments, delivery devices 100′″ and/or 100″″ comprise an agent similar to agent 200A within delivery device 100″ (e.g. insulin). In other embodiments, delivery devices 100′″ and/or 100″″ comprise an agent dissimilar to agent 200A within delivery device 100″.

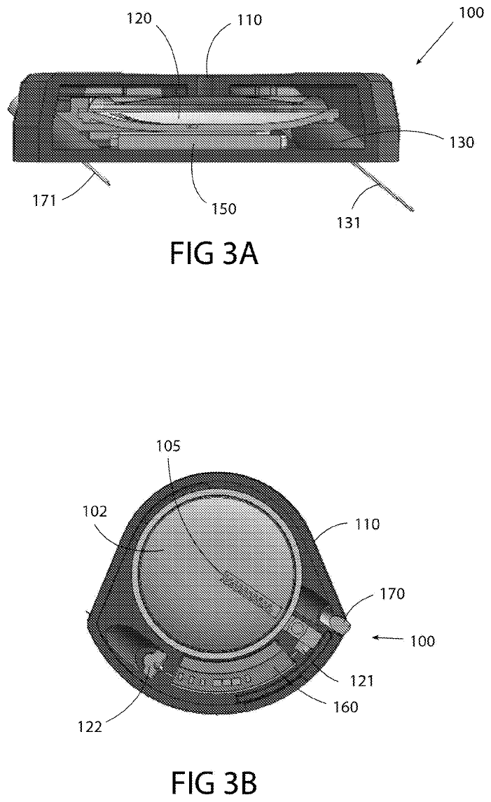

In some embodiments, system 10 and/or its components of FIG. 1, FIG. 2A, and/or 2B are constructed and arranged as described herebelow in reference to FIGS. 3-24.

Referring now to FIGS. 3A-B, side and top sectional views, respectively, of a delivery device are illustrated, consistent with the present inventive concepts. Delivery device 100 of FIG. 3A-B includes housing 110 which surrounds reservoir 120 and other components as shown. Reservoir 120 can comprise a relatively flat, circular geometry, and can be positioned at a relatively central location within housing 110. Pumping mechanism 160 can be positioned on the periphery of reservoir 120 as shown. Power supply 150 can be positioned under reservoir 120 as shown. TDA 130 can include cannula 131, shown in a deployed position (e.g. a position in which cannula 131 has penetrated the patient's skin into subcutaneous tissue or other internal body location).

Device 100 can include an integrated sensing assembly 170, such as an integrated glucose sensing assembly or other patient-parameter sensing assembly as described herein. Sensing assembly 170 can comprise cannula 171, shown in a deployed position in FIG. 3A (e.g. a position in which cannula 171 has penetrated the patient's skin into subcutaneous tissue or other internal body location).

Referring now to FIGS. 4A-E, various views of a reservoir, reservoir analysis, and a reservoir-forming tool are illustrated, consistent with the present inventive concepts. In FIG. 4A, delivery device 100 comprises a portion of housing 110 that provides a base-portion of reservoir 120, base portion 123. Reservoir 120 can comprise a reservoir comprising a “rolling sock” construction, such that a top portion 124 of reservoir 120 comprises a rolling film construction, such as a construction in which top portion 124 comprises a rolling sock 124 that unfurls along one or more bends, and a center portion of reservoir 120 expands upward as reservoir 120 is filled. Base portion 123 can have a rigid construction. Base portion 123 can be constructed of cyclic olefin copolymer (COC) and can include a film covering, such as a two-layer film covering comprising polychlorotrifluoroethylene (PCTFE), such that the film faces the internal portion of reservoir 120 (e.g. becomes the surface in contact with agent 200 within reservoir 120, such as when agent 200 comprises insulin). In some embodiments, COC material of the two-layer film faces agent 200. In some embodiments, base portion 123 comprises a different agent 200 contacting surface material (e.g. different covering) that (similar to PCTFE) provides: compatibility with insulin; high moisture barrier properties; and/or oxygen barrier properties. Top portion 124 can be fixedly attached to base portion 123 via ultrasonic and/or thermal welding, and/or via the use of adhesives. Reservoir 120 can comprise a circular shape, as shown, and can have a diameter of less than or equal to 40 mm, or 35 mm, and a height (e.g. when filled) of less than or equal to 7.5 mm, or 6.5 mm.

FIG. 4B illustrates an analysis of stress on reservoir 120 as fluid is extracted from the reservoir 120 causing the top portion 124 to alter its configuration.

FIG. 4C illustrates pressure within reservoir 120 at different fill volumes.

FIG. 4D illustrates forming tool 700b configured to form a film-based reservoir 120, such as a vacuum-forming and/or heat-forming tool.

FIG. 4E illustrates a formed top made using forming tool 700b as an exemplar of a flexible top portion 124.

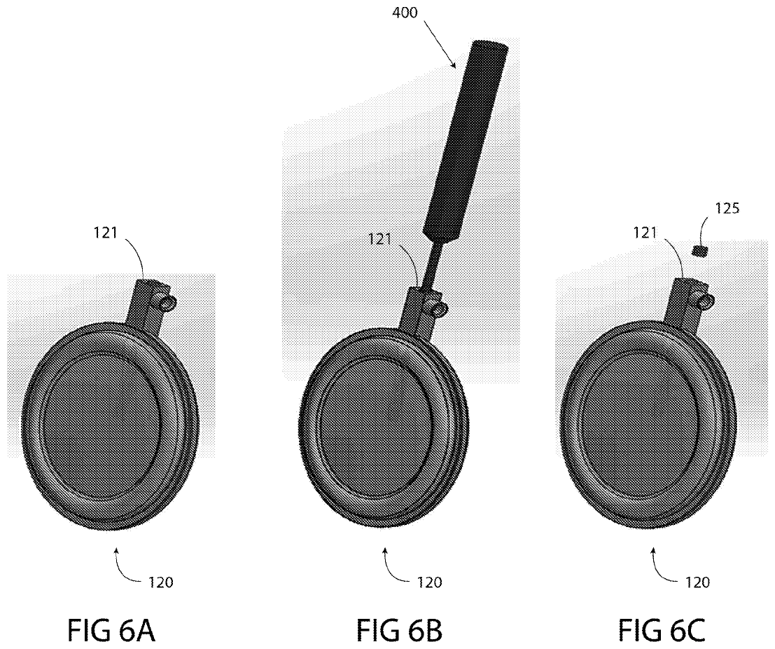

Referring now to FIG. 5, a sectional view of a portion of a delivery device is illustrated, consistent with the present inventive concepts. Shown in FIG. 5 are reservoir 120 and bubble capture element 105 of delivery device 100. Bubble capture element 105 can be positioned proximate reservoir 120 as shown (e.g. on a solid portion of reservoir 120). In some embodiments, bubble capture element 105 is integrated into a solid portion of reservoir 120. Reservoir 120 comprises fill port 121 and drain port 122. Drain port 122 can be connected (e.g. via one or more tubes 101 not shown) to pumping mechanism 160. Fill port 121 can be sealed after filling (e.g. sealed with a plug, such as plug 125 described in reference to FIGS. 6A-C). The plug 125 can be passed into fill port 121 through fill device 400, for example while agent 200 is still in continuous contact with both reservoir 120 and fill device 400 or after that continuous contact has been interrupted. In some embodiments, multiple reservoirs (e.g. at least 12) are filled (e.g. with agent 200) simultaneously in the manufacture of multiple delivery devices 100. In some embodiments, reservoir 120 (e.g. and other portions of device 100, such as at least all fluid pathways and cannula 131) are sterilized, such as a sterilization performed prior to an aseptic filling of reservoir 120 (e.g. filling with agent 200).

Referring now to FIGS. 6A-C, sequential views of a procedure for filling a reservoir are illustrated, consistent with the present inventive concepts. Reservoir 120 comprises fill port 121. In FIG. 6A, reservoirs 120 are positioned and oriented for a subsequent filling procedure.

In FIG. 6B, a fill device 400 is operably (e.g. at least fluidly) attached to fill port 121 of reservoir 120. During the fill (e.g. with agent 200), vacuum can be applied to reservoir 120 to assist in the filling, such that no gas needs to be displaced from reservoir 120 during the fill process.

In FIG. 6C, fill device 400 has been removed, and a plug 125 can be inserted into fill port 121. In some embodiments, plug 125 comprises a septum that is in place during and/or after the fill process, such as a septum that maintains a seal within fill port 121, prior to, during, and after reservoir 120 being filled.

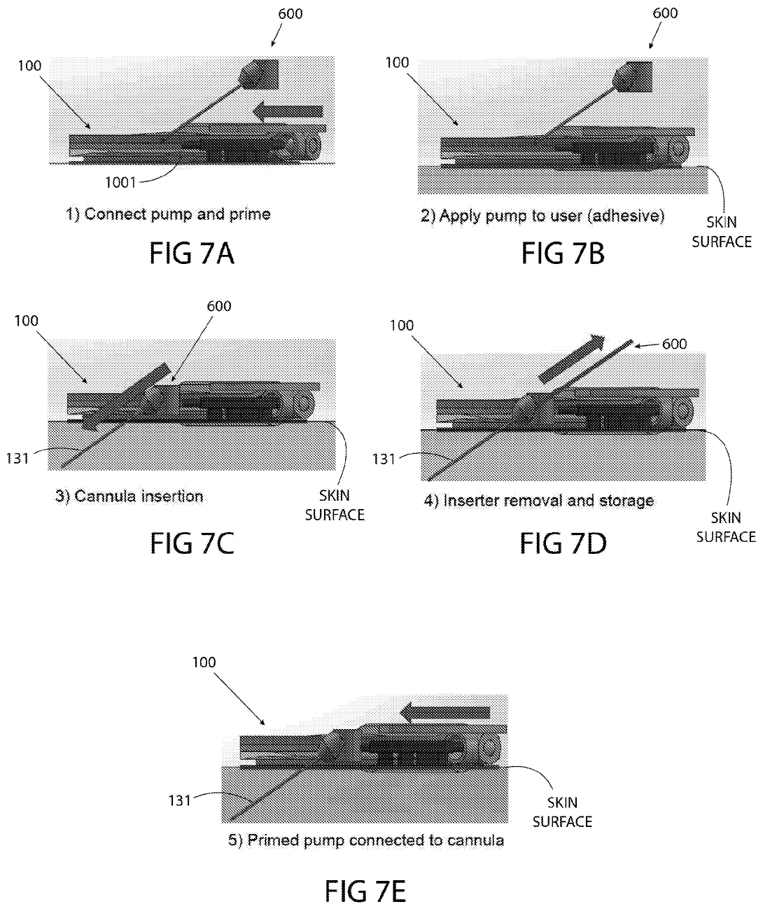

Referring now to FIGS. 7A-E, views of a procedure for priming a delivery device and introducing an agent delivery cannula into a patient using an insertion device are illustrated, consistent with the present inventive concepts. Insertion device 600 can be configured to insert one or more cannula 131 of TDA 130 into a patient.

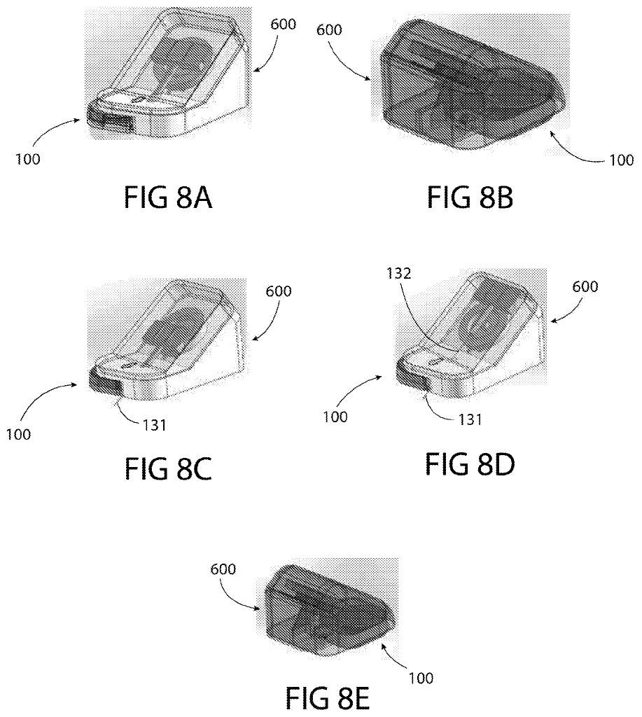

In FIG. 7A, insertion device 600 is operably attached to a delivery device 100. In some embodiments, insertion device 600 isn't attached to delivery device 100 until the step illustrated in FIG. 7C. In the step of FIG. 7A, reservoir 120 undergoes a first rotation that causes pumping mechanism 160 to become fluidly connected to reservoir 120 (e.g. a needle of pumping mechanism 160 pierces a septum of drain port 122 of reservoir 120, or a needle of drain port 122 pierces a septum of mechanism 160). This first rotation can be performed by insertion device 600 (e.g. as described in reference to FIG. 8B) and/or it can be performed manually by an operator of system 10. This first rotation can further cause delivery device 100 (e.g. control module 140) to enter an “on” state. In this on state, delivery device 100 can perform a first communication (e.g. a transfer of data) with communication device 300. This first rotation can further cause a priming of one or more flow pathways of delivery device 100 (e.g. a priming of flow pathways including pathways of pumping mechanism 160 with agent 200 provided by reservoir 120). In some embodiments, delivery device 100 comprises pad 1001 positioned proximate drain port 122, where pad 1001 can be configured to collect fluid such as “froth” (e.g. a mixture of an aqueous or other liquid formulation of an agent, such as insulin or glucagon, and a gas such as air) that may be produced during the priming of the flow pathways.

In the step illustrated in FIG. 7B, delivery device 100 is adhered to the patient's skin (e.g. via skin attachment assembly 111).

In the step illustrated in FIG. 7C, insertion device 600 is attached (if not previously attached), and insertion device 600 is activated such that cannula 131 is advanced through the skin of the patient (e.g. into subcutaneous tissue or other internal body location). In some embodiments, both a needle 132 and surrounding cannula 131 are inserted through the skin of the patient.

In the step illustrated in FIG. 7D, insertion device 600 retracts needle 132 (if present) from within the patient (e.g. retracted within cannula 131 such that only cannula 131 remains passed through the skin of the patient). In some embodiments, insertion device 600 automatically retracts needle 132 (e.g. without further operator action). In some embodiments, needle 132 is removed from delivery device 100 by device 600 (e.g. and safely enclosed within device 600).

In the step illustrated in FIG. 7E, reservoir 120 and pumping mechanism 160 undergo, in unison, a second rotation that causes pumping mechanism 160 to be fluidly connected with TDA 130 (e.g. a needle of pumping mechanism 160 pierces a septum on an input portion of TDA 130, and/or a needle of an input portion of assembly 130 pierces a septum of mechanism 160). This second rotation can be performed by insertion device 600 and/or it can be performed manually by an operator of system 10. TDA 130 can then be primed (e.g. automatically primed) with fluid (e.g. with agent 200 provided by reservoir 120 and propelled by pumping mechanism 160).

Insertion device 600 can be detached from delivery device 100 after the step of FIG. 7D (e.g. prior to the step of FIG. 7E), or after the step of FIG. 7E.

Referring now to FIGS. 8A-E, views of a procedure for priming a delivery device and introducing an agent delivery cannula into a patient using an insertion device are illustrated, consistent with the present inventive concepts. Insertion device 600 can be configured to insert one or more cannula 131 of TDA 130 into a patient. The operations performed as illustrated in FIG. 8A-E can be similar to those described in reference to FIGS. 7A-E hereabove.

In FIG. 8A, insertion device 600 is operably connected with delivery device 100. Cannula 131 of delivery device 100 is in an undeployed state. Two drive mechanisms (e.g. spring-driven drive mechanisms) of insertion device 600 are in a “ready to deploy” state.

In the step illustrated in FIG. 8B, a first control 601a (e.g. a button) of insertion device 600 can be activated to cause reservoir 120 to undergo a first rotation. In the step of FIG. 7B, the first rotation causes pumping mechanism 160 to become fluidly connected to reservoir 120 (e.g. a needle of pumping mechanism 160 pierces a septum of drain port 122 of reservoir 120, and/or a needle of drain port 122 pierces a septum of mechanism 160). In an alternative embodiment, the first rotation can be performed manually by an operator of system 10. This first rotation can further cause delivery device 100 (e.g. control module 140) to enter an “on” state. In this on state, delivery device 100 can perform a first communication (e.g. a transfer of data) with communication device 300. This first rotation can further cause a priming of one or more flow pathways of delivery device 100 (e.g. a priming of flow pathways including pathways of pumping mechanism 160 with agent 200 provided by reservoir 120). In some embodiments, delivery device 100 comprises a pad (not shown but such as a pad positioned proximate drain port 122), where the pad is configured to collect “froth” (as described herein) that may be produced during the priming of the flow pathways. In some embodiments, a second control 601b (e.g. a button) of insertion device 600 becomes activated (e.g. unlocks, is connected, and/or otherwise becomes functional) after the first rotation is performed.

In the step illustrated in FIG. 8C, delivery device 100 is prepared for application to the patient (e.g. a cover positioned on skin attachment assembly 111 is removed). Subsequently, delivery device 100 is positioned on the patient's skin (e.g. on an arm, leg, or abdomen of the patient).

In the step illustrated in FIG. 8D, the second control 601b is then activated and cannula 131 is advanced through the skin of the patient (e.g. into subcutaneous tissue or other internal body location). In some embodiments, both a needle 132 and surrounding cannula 131 are inserted through the skin of the patient. Subsequently, insertion device 600 retracts needle 132 (if present) from within the patient (e.g. retracted within cannula 131 such that only cannula 131 remains passed through the skin of the patient). In some embodiments, needle 132 is removed from delivery device 100 by device 600 (e.g. and safely enclosed within device 600).

In the step illustrated in FIG. 8E, with the cannula 131 in place within the patient, insertion device 600 performs a second rotation that causes pumping mechanism 160 to be fluidly connected with TDA 130 (e.g. a needle of pumping mechanism 160 pierces a septum on an input portion of TDA 130, or a needle of assembly 130 pierces a septum of mechanism 160). This second rotation can be performed by insertion device 600 and/or it can be performed manually by an operator of system 10 (e.g. after insertion device 600 has been detached from delivery device 100). TDA 130 can then be primed (e.g. automatically primed) with fluid (e.g. a precise amount of agent 200 is provided by reservoir 120 and propelled by pumping mechanism 160 to fill assembly 130). In some embodiments, the second rotation cannot be performed unless needle 132 has been removed from cannula 131 (e.g. step of FIG. 8E is delayed until needle 132 is withdrawn, such as an automatic withdrawal of needle 132 by device 100)>

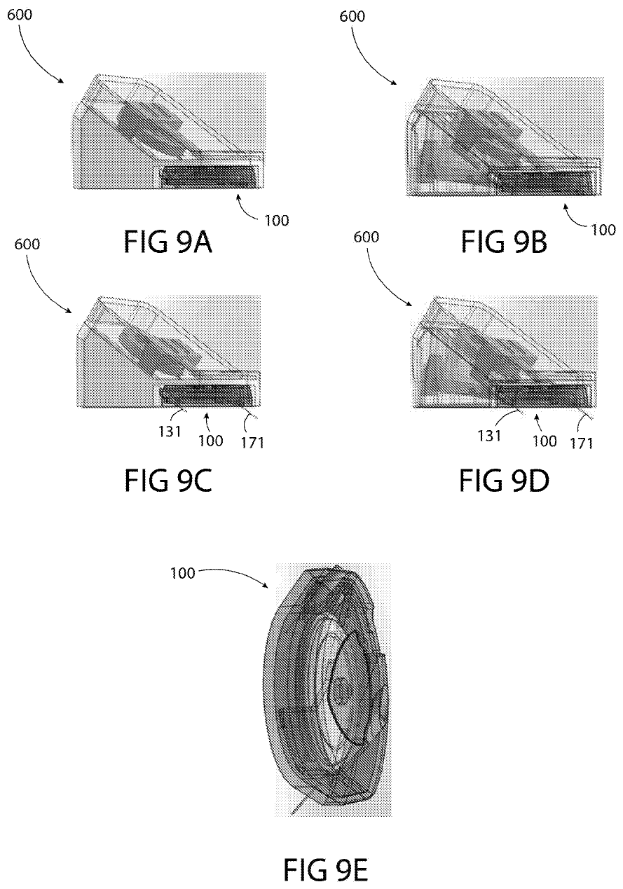

Referring now to FIGS. 9A-E, views of a procedure for introducing a cannula of a sensing assembly into a patient using an insertion device are illustrated, consistent with the present inventive concepts. Insertion device 600 can be configured to insert one or more cannula 171 of sensing assembly 170 thru a patient's skin and into the patient (e.g. into the subcutaneous tissue and/or other internal body location).

In FIGS. 9A-B, insertion device 600 is operably engaged with delivery device 100 and delivery device 100 is positioned on the patient's skin.

In FIGS. 9C-D, a control of insertion device 600 is activated (e.g. control 601 described herein) and at least cannula 171 of sensing assembly 170 is inserted into the patient, such as in a similar arrangement as described in reference to inserting cannula 131 as described in reference to FIGS. 7A-E and/or 8A-E.

Once cannula 171 is inserted into the patient, sensing assembly 170 can be operably connected (e.g. at least electrically connected) to control module 140. Once cannula 171 is inserted into the patient, a seal can be formed between cannula 171 and housing 110.

Referring now to FIGS. 10A-B, various views of a pumping mechanism are illustrated, consistent with the present inventive concepts. In FIG. 10A, a perspective transparent view of pumping mechanism 160 is shown. In FIG. 10B, a side sectional view of pumping mechanism 160 is shown illustrating microfluidic channels. Pumping mechanism 160 can comprise a MEMS-based assembly. Pumping mechanism 160 can comprise a composite assembly of one or more silicon sensor and actuators that are assembled using MEMS assembly techniques on top of a microfluidic substrate, connection layer 162, mounted on an integrated circuit board, board 16001. Pumping mechanism 160 can comprise the curved geometry shown, such as to provide efficient packaging of pumping mechanism 160 and other components of delivery device 100 within housing 110 (e.g. a circular shaped housing 110) and, for example, to facilitate rotation of the pumping mechanism 160 around a central axis within the housing 110.

Pumping mechanism 160 comprises connection layer 162, such as a microfluidic routing layer including the microfluidic channels 162a-f shown in FIG. 10B. Mechanism 160 can include: inflow needle 16002; sensor 16003; actuator 16004; sensor 16005; valve 16006; valve 16007; valve 16008; sensor 16009; and outflow needle 16010; each as shown in FIG. 10A. Inflow needle 16002 can comprise a needle or other component configured to fluidly attach pumping mechanism 160 to reservoir 120 (e.g. to pierce a septum, valve or other penetrable component of reservoir 120, such as is described herein). Sensor 16003 can comprise a pressure sensor positioned to measure pressure within or at least proximate reservoir 120. Actuator 16004 can comprise a MEMS pumping actuator configured to propel a fluid, such as agent 200. Actuator 16004 can comprise one or more pumping elements (e.g. pumping diaphragms, such as diaphragm 16017 described herein) and one or more valves. Sensor 16005 can comprise a flow sensor. Valve 16006 can comprise a passive check valve that is configured to prevent runaway (forward) flow in a failure state (e.g. a failure state of actuator 16004). Valve 16007 can comprise an active check valve that is redundant with valves of actuator 16004. Valve 16008 can comprise a passive check valve configured to prevent backflow of fluid (e.g. backflow of agent 200). Sensor 16009 can comprise a pressure sensor positioned to measure pressure within or at least proximate cannula 131. Outflow needle 16010 can comprise a needle or other component configured to fluidly attach pumping mechanism 160 to TDA 130 (e.g. to pierce a septum, valve, or other penetrable component of assembly 130, such as is described herein). The assembly can be covered with a package cap, cap 16018 shown, and filled or sealed with a filling material such as epoxy or fluorosilocone gel.

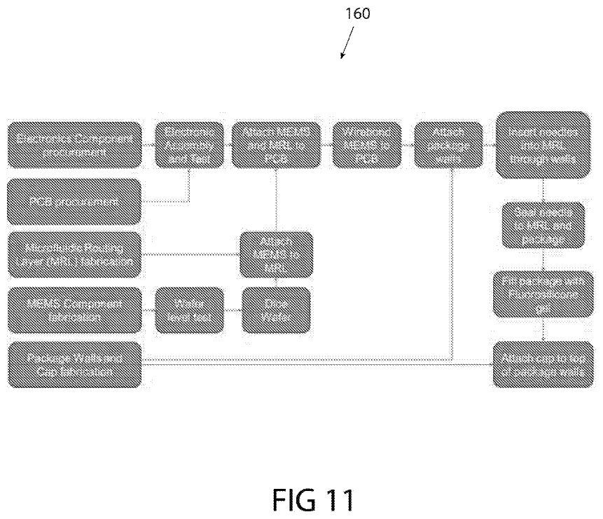

Referring now to FIG. 11, a flow chart of a fabrication process of a pumping mechanism is illustrated, consistent with the present inventive concepts. Pumping mechanism 160 can comprise a composite device designed and configured to maximize yield. MEMS components can be tested first at the wafer level, and then individually before being selected for inclusion in pumping mechanism 160, minimizing the likelihood that mechanism 160 will fail due to failure of one of the individual components.

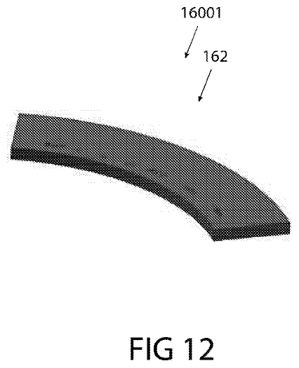

Referring now to FIG. 12, a fluidic connection layer including a substrate is illustrated, consistent with the present inventive concepts. Substrate 16001 can comprise a printed circuit board (PCB). Connection layer 162 can be attached to the PCB by an adhesive such as an epoxy. Substrate 16001 can include conductive traces to electrically connect components to each other and to the rest of device 100 (e.g. using solder). MEMS components of pumping mechanism 160 can be wirebonded from pads on the MEMS device to substrate 16001. Individual electrical components, not shown but typically integrated circuits, capacitors, resistors, and the like, can also be assembled onto substrate 16001.

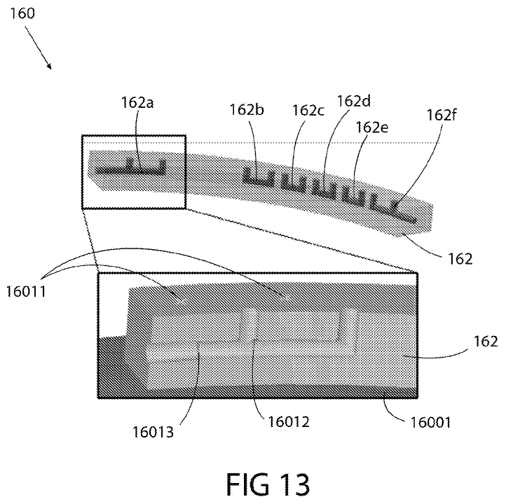

Referring now to FIG. 13, a sectional and a magnified view of a portion of a pumping mechanism including a microfluidic routing layer is illustrated, consistent with the present inventive concepts. As shown in FIG. 13, connection layer 162 can be fixedly attached (e.g. adhesively attached) to substrate 16001.

Connection layer 162 can comprise one or more fiducial marks, such as marks 16011 shown. Marks 16011 can be used to align one or more MEMS components during the fabrication of pumping mechanism 160.

Connection layer 162 can comprise features for seating one or more needles, feature 16013. Feature 16013 can comprise a projection and/or a reduced diameter portion which prevents translation of a needle inserted within a fluid path (e.g. needles 16002 and/or 16010 described herein).

Connection layer 162 can comprise one or more corners that include a turn (e.g. a 90° turn) in a flow path that includes a filleted corner, corner 16012 shown, such as a filleted or other rounding of the corner to reduce shear, reduce turbulence, and/or otherwise provide enhanced flow dynamics (e.g. to minimize damage to insulin and/or fluids being propelled around the corner). In some embodiments, all corners of connection layer 162 comprise filleted corners.

Connection layer 162 can comprise a material such as COC and/or polymethyl methacrylate. Connection layer 162 can comprise a hydrophilic coating. Connection layer 162 can be insertion molded, such as when two insertion molded parts are produced and bonded together to make connection layer 162. MEMS components can be “picked and placed”, and then bonded with a die attach adhesive (DAA).

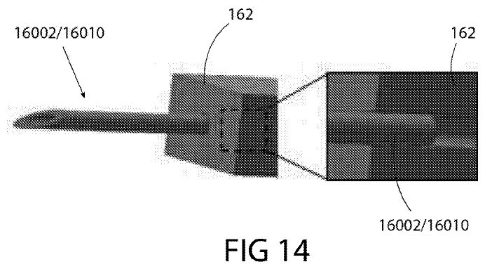

Referring now to FIG. 14, a perspective and a magnified sectional view of a fluid path connecting element is illustrated, consistent with the present inventive concepts. Pumping mechanism 160 can comprise one or more fluid path connecting elements that are configured to create a fluid path between pumping mechanism 160 and reservoir 120 and between pumping mechanism 160 and TDA 130, each as described herein. These fluid path connections can be made when one or more portions of delivery device 100 are rotated, also as described herein. FIG. 14 shows a fluid path connecting element, such as inflow needle 16002 and/or outflow needle 16010, each as described herein. In some embodiments, needles 16002 and/or 16010 comprise a length of approximately 5 mm, with at least 1 mm (e.g. approximately 3 mm) extending out of connection layer 162. Needles 16002 and/or 16010 can comprise a diameter of at least 30Ga, such as 28Ga. Needles 16002 and/or 16010 can be positioned against a microfluidic layer feature (e.g. feature 16013 described herein and shown in FIG. 13), such as to prevent slippage while puncturing a septum of device 100 during a fluidic connection step. In some embodiments, needles 16002 and/or 16010 are fixedly attached within a fluid path, such as with an adhesive or compression fit.

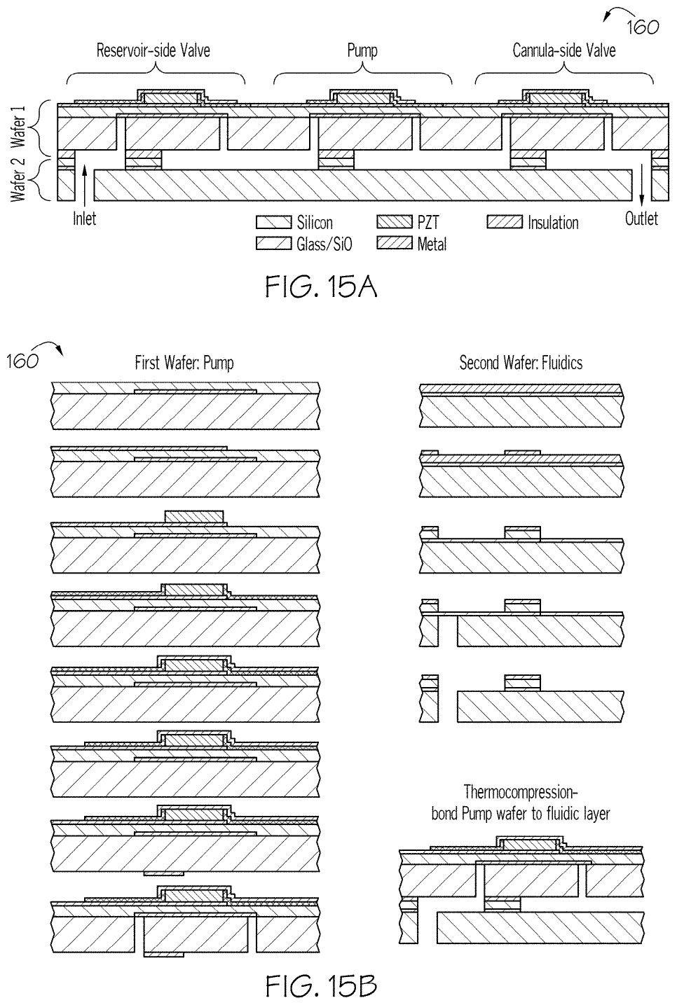

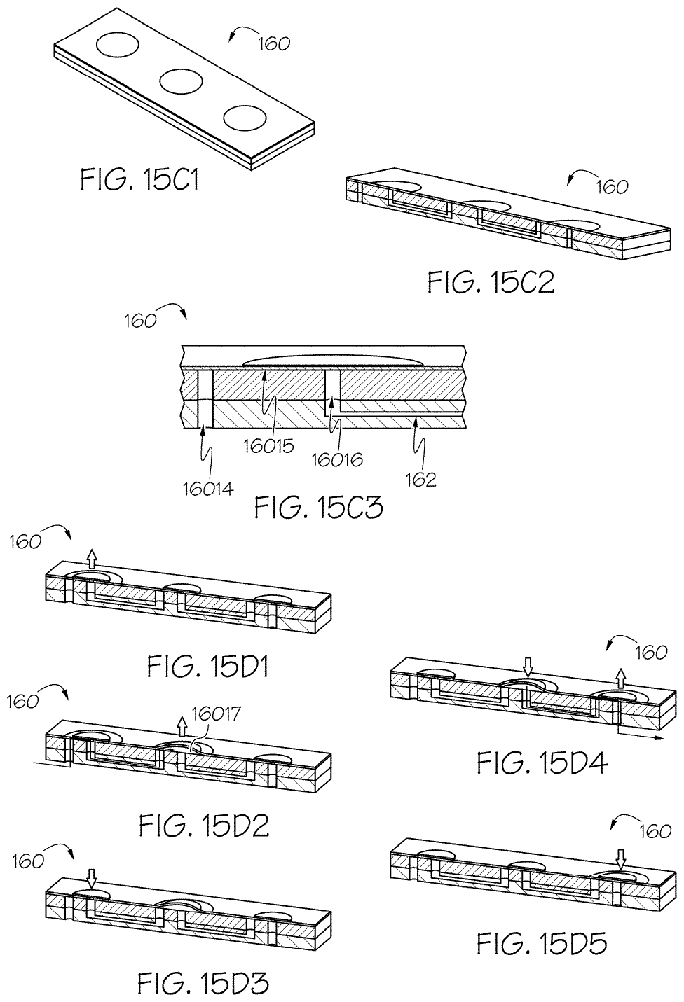

Referring now to FIGS. 15A-D, a pumping actuator schematic, a series of manufacturing steps for a pumping actuator, a set of three pumping actuator views, and a series of pump activation steps, respectively, are illustrated, consistent with the present inventive concepts. Pumping mechanism 160 of FIGS. 15A-D can comprise a pumping actuator pump 161 that includes a silicon diaphragm, diaphragm 16017, that is actuated using micro-machined PZT film. Mechanism 160 can include three chambers: an active valve that regulates flow on the input side, pump 161, and an active valve that regulates flow on the output side.

FIG. 15A is a schematic view of pumping mechanism 160 components.

FIG. 15B shows a series of steps for manufacturing a portion of pumping mechanism 160. Two wafers can be fabricated separately, then bonded together.

FIG. 15C shows a perspective view, perspective sectional view, and a side sectional view of pumping mechanism 160. Pumping mechanism includes inlet 16014, chamber 16015, outlet 16016 (e.g. an outlet to another chamber), and connection layer 162, each positioned and fluidly connected as shown.

FIG. 15D shows a pump activation sequence. In Step 1, a first chamber is activated (e.g. opened for fluid flow therethrough), creating a fluid path between reservoir 120 and a second chamber, chamber 16017 shown. In Step 2, chamber 16017 is activated, drawing fluid from reservoir 120 to second chamber 16017. In Step 3, the first chamber is deactivated (e.g. preventing fluid flow therethrough) to close the fluid path. In Step 4, chamber 16017 is deactivated, and fluid within chamber 16017 is at a positive pressure, while simultaneously a third chamber is activated, creating a flow path between pump 161 and TDA 130. Finally in Step 5, the third chamber is deactivated, forcing the intended amount of fluid into assembly 130.

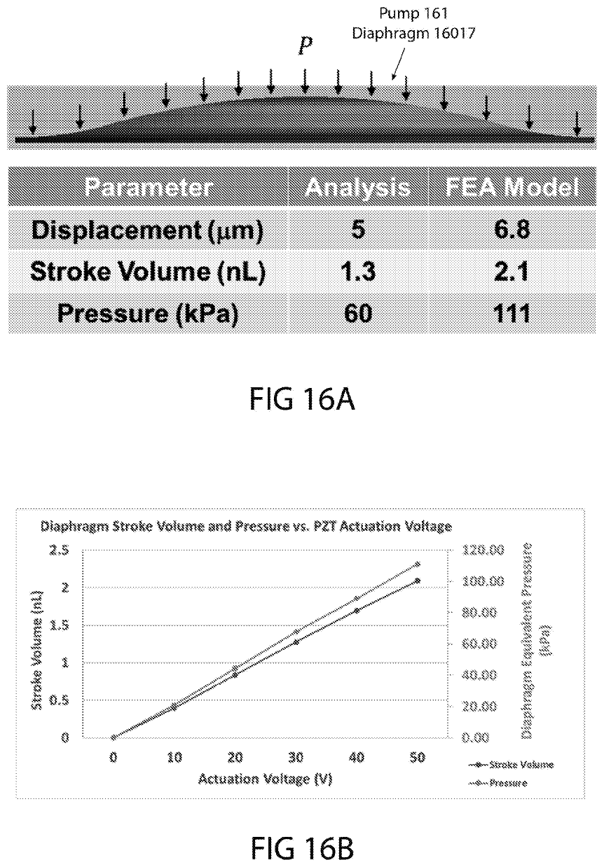

Referring now to FIGS. 16A-B, results of a finite element analysis (FEA) on a pumping mechanism are illustrated, consistent with the present inventive concepts. Pump 161 and other components of pumping mechanism 160 are constructed and arranged to provide precision in certain pumping parameters, such as stroke volume, compression ratio, membrane displacement, and output pressure. FIG. 16A illustrates a portion of pump 161, which can include a chamber volume of 10.6 nL. Pump 161 can include a diaphragm, diaphragm 16017 shown, which can comprise: a diameter of 1.06 mm; and/or a thickness of 10 μm. Pump 161 can be configured to: displace diaphragm 16017 a distance of 5 μm; and/or provide a stroke volume of 1.3 nL. Pump 161 can provide an output pressure of at least 60 kPa. Actuation voltage of pump 161 can be linearly related to stroke volume and output pressure, as shown in FIG. 16B.

Referring now to FIGS. 17A-B, a perspective view and an assembly process, respectively, of an embodiment of a flow sensor is illustrated, consistent with the present inventive concepts. In some embodiments, flow sensor 16005 of pumping mechanism 160, and/or another flow sensor of device 100 comprise a hot wire anemometer as described and illustrated in reference to FIGS. 17A-B. The sensor of FIG. 17A can comprise two unheated wires 16005a, a heated wire 16005b, and electrical connection pads 16005c. The sensor can be operated with constant-temperature controlled by a constant-resistance analog circuit. Wires 16005a and 16005b allows for direct-flow, calorimetric and time-of-flight configurations. Fluid flows past the first wire 16005a, is heated by the second wire 16005b, then flows past the third wire 16005a. The difference in temperature-based resistance of the unheated wires allows calculation of the flow rate. The fabrication process of the sensor involves two separate wafers bonded and then further etched, as shown in FIG. 17B.

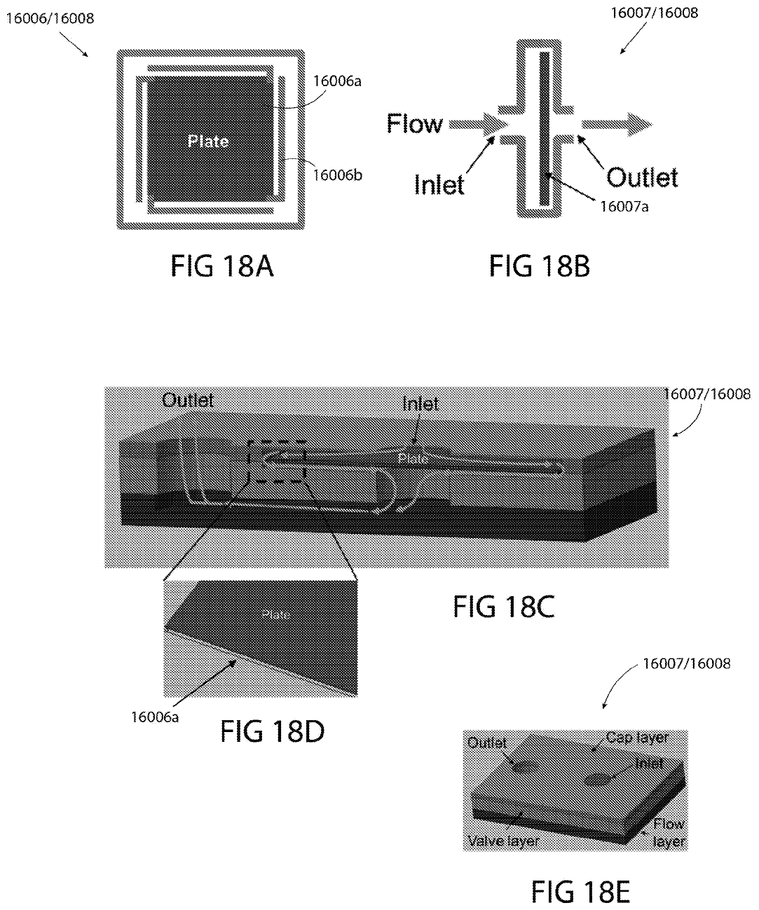

Referring now to FIGS. 18A-E, various views of an embodiment of a passive check valve is illustrated, consistent with the present inventive concepts. In some embodiments, valve 16006 of pumping mechanism 160, valve 16008 of pumping mechanism 160, and/or another valve of device 100 comprise a passive check valve as described and illustrated in reference to FIGS. 18A-E. Valves 16006/16008 can be configured to close at a high fluid flow rate, such as to prevent overdosing (e.g. due to malfunction such as a crushed reservoir) and/or to prevent backflow from TDA 130.

FIGS. 18A-B are top and side sectional views of an active check valve including plate 16006a and springs 16006b. Plate 16006a is suspended in the fluid pathway on springs 16006b that float the plate to allow fluid to flow around plate 16006a during low flow periods, but allow plate 16006a to move forward (e.g. to cover an outlet) during high flow periods. For backflow prevention, springs 16006b are adjusted such that the valve 16006/16008 is normally closed unless opened by forward pressure.

FIG. 18C is a side sectional view of the active check valve showing the direction of flow. FIG. 18D is a perspective view of plate 16006a. FIG. 18E is a sectional view showing various portions of valve 16006/16008.

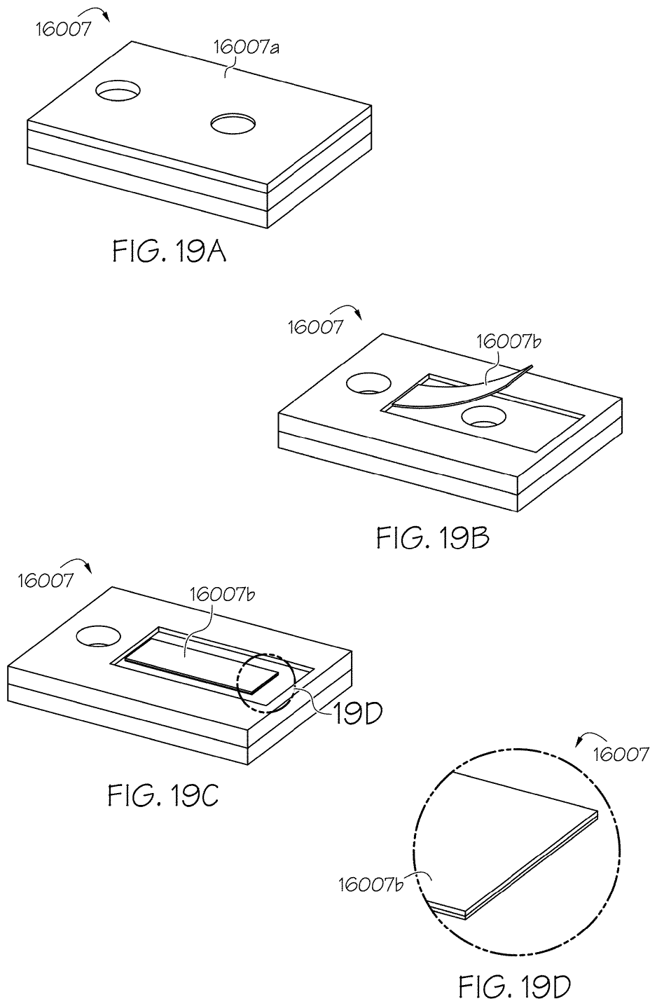

Referring now to FIGS. 19A-D, various views of an embodiment of an active check valve is illustrated, consistent with the present inventive concepts. In some embodiments, valve 16007 of pumping mechanism 160, and/or another valve of device 100 comprise an active check valve as described and illustrated in reference to FIGS. 19A-D. Valve 16007 of FIGS. 19A-D comprises a three-layer design, and it can include a bimorph construction that creates a voltage-dependent valve flap. Valve 16007 can be included in pumping mechanism 160 to provide a redundancy with other included valves. For example, valve 16007 can be included to prevent undesired forward flow (e.g. in a failure condition).

FIG. 19A is a perspective sectional view showing various portions of valve 16007, including cap layer 16007a. FIG. 19B is a sectional view, with cap layer 16007a removed, showing bimorph 16007b activated to cause its deflection (e.g. shown with an exaggerated amount of deflection). FIG. 19C is a perspective view with bimorph 16007b in an unactivated, undeflected state. FIG. 19D is a close-up view of the two layers of bimorph 16007b.

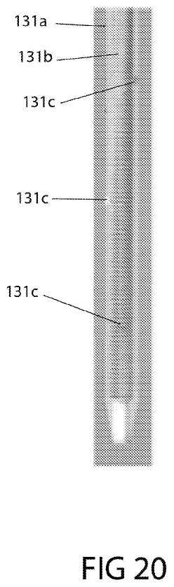

Referring now to FIG. 20, a side view of a cannula is illustrated, consistent with the present inventive concepts. Cannula 131 of FIG. 20 comprises a shaft, shaft 131a, a reinforcing coil, coil 131b (e.g. a metal or polymeric reinforcing coil), and multiple (three shown) side holes 131c. Cannula 131 can be inserted into the patient, as described herein, and remain in place (e.g. continuously or intermittently delivering agent 200 to the patient) for a period of at least 3 days, or at least 7 days.

Referring now to FIG. 21, a schematic view of a communication architecture for an agent delivery system is illustrated, consistent with the present inventive concepts. As shown in FIG. 21, system 10 can include a first delivery device 100a, a communication device 300 (e.g. a cell phone of the patient), and network 800. In some embodiments, system 10 further includes a second delivery device 100b, such as when first delivery device 100a delivers a first agent 100a (e.g. insulin), and second delivery device 100b delivers a second agent 100b (e.g. glucagon and/or glucose). In an alternative embodiment, delivery device 100 comprises a device configured to deliver (e.g. independently deliver) two separate agents, such as is described in reference to delivery device 100′ of FIG. 2. In some embodiments, system 10 further includes sensing device 500 (as shown), and/or delivery devices 100a and/or 100b include an integrated sensing assembly 170 as described herein.

Delivery device 100 can be operably connected (e.g. wirelessly connected) to communication device 300 (e.g. a cell phone of the patient), such that information can be transferred from delivery device 100 to network 800 (e.g. the cloud) via device 300. This information transfer can be relatively continuous and/or it can take place intermittently (e.g. once an hour, once every four hours, once every eight hours, and/or once per day). For example, delivery device 100 can be configured to function autonomously, for example when device 300 or network 800 are not available. In some embodiments, communication device can be configured to receive information (e.g. from the patient) regarding actual or planned: meals; activity; glucose level or other physiologic parameter levels; and other patient-related information. In some embodiments, network 800 is configured to utilize machine learning on the data it receives, such as to optimize algorithms 141 of control module 140 of device 100. Via connectivity to network 800, system 10 can perform data backup, communications with care givers, and/or cross-patient machine learning (e.g. to support further enhancements of algorithms 141).

In some embodiments, system 10 communication protocols will be at least Bluetooth LE 4.1, which is approved by the NSA as FIPS compliant for secure communications.

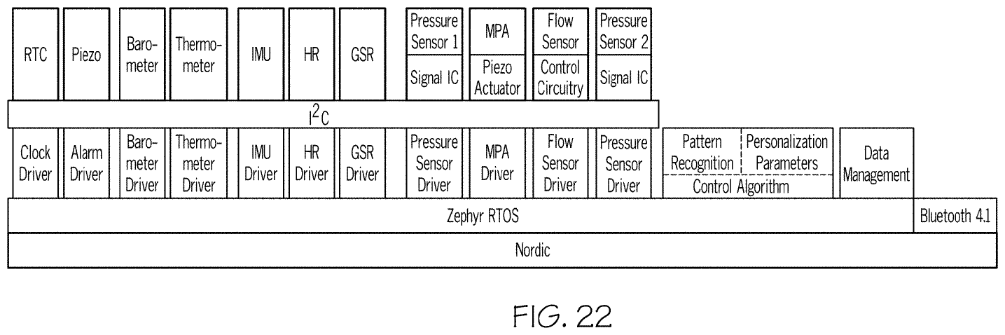

Referring now to FIG. 22, a schematic view of a delivery device is illustrated, consistent with the present inventive concepts. Delivery device 100 of FIG. 22 includes various components as shown. Components designated with black boxes with white lettering are hardware components; the rest are software components. Delivery device 100 can include a Nordic, Bluetooth-enabled nrf52 class microprocessor. Software drivers and control modules can be implemented on a Zephyr Real Time Operating System. Functional element 199 can comprise an onboard inertial measurement unit (IMU), that can provide motion data which can be translated into activity data (e.g. as determined by a motion pattern recognition component of algorithm 141). Controller 140 can include a hardware watchdog timer that can protect against system failure by enabling automatic rebooting of the controller 140 (e.g. a microprocessor of controller 140) should a critical problem occur.

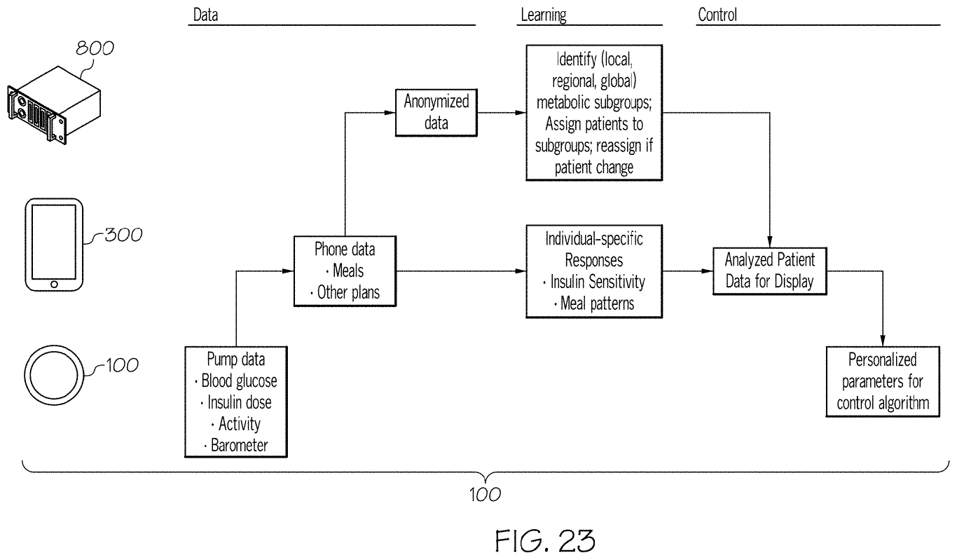

Referring now to FIG. 23, a schematic view of a delivery system and a flow chart of a learning architecture for the delivery system is illustrated, consistent with the present inventive concepts. As shown in FIG. 23, system 10 can include delivery device 100, communication device 300, and network 800, each as described herein. Information can be provided by delivery device 100 and/or other components of system 10, to network 800, such as via communication device 300, also as described herein.

Algorithm 141 can be configured for closed loop control of insulin delivery for a Type 1, Type 2, and/or other diabetic patient, such as when configured as an artificial pancreas. Algorithm 141 may include, but not require, the processing of meal and exercise data (e.g. when available). Enhancements (e.g. learning) to algorithm 141 by system 10 can take place on communication device 300 (e.g. a cell phone) and/or network 800 (e.g. in the cloud). For insulin delivery, system 10 can identify patient-specific parameters (e.g., insulin sensitivity and/or hydration/Vd) that affect blood glucose (BG) control and that can be transmitted to device 100 to enhance algorithm 141. Metabolic subgroups (e.g., “brittle,” or “hyperhormonal”) can be identified, for whom different control parameters may be appropriate, and algorithm 141 can be modified based on data-driven patient assignments to these groups. Control parameters can be downloaded to each new device 100, for example when it is initialized by the patient. System 10 can include a layer to protect against pattern-recognition misclassifications. FIG. 23 outlines a particular learning architecture of system 10.

Referring now to FIG. 24, a schematic view of a delivery system is illustrated, consistent with the present inventive concepts. System 10 can be configured as a glycemic control system. As shown in FIG. 24, system 10 can include a first delivery device 100a, a communication device 300 (e.g. a cell phone of the patient), and network 800. In some embodiments, system 10 further includes a second delivery device 100b, such as when first delivery device 100a delivers a first agent 200a (e.g. insulin), and second delivery device 100b delivers a second agent 200b (e.g. glucagon and/or glucose). In an alternative embodiment, delivery device 100 comprises a device configured to deliver (e.g. independently deliver) two separate agents 200, such as is described in reference to delivery device 100′ of FIG. 2A. In some embodiments, system 10 further includes sensing device 500 as shown, and/or delivery devices 100a and/or 100b include an integrated sensing assembly 170 as described herein.

Delivery device 100 can be operably connected (e.g. wirelessly connected) to communication device 300 (e.g. a cell phone of the patient), such that information can be transferred from delivery device 100 to network 800 (e.g. the cloud) via device 300. This information transfer can be relatively continuous and/or it can take place intermittently (e.g. once per hour, once every four hours, once every eight hours, and/or once/day). For example, delivery device 100 can be configured to function autonomously, for example when device 300 or network 800 are not available. In some embodiments, communication device 300 can be configured to receive information (e.g. from the patient) regarding actual or planned: meals; activity; glucose level or other physiologic parameter levels; and other patient-related information. In some embodiments, network 800 is configured to utilize machine learning on the data it receives, such as to optimize algorithms 141 of controller 140 of device 100. Via connectivity to network 800, system 10 can perform data backup, communications with care givers, and/or cross-patient machine learning (e.g. to support further enhancements of algorithms 141).

System 10 can further include a fluid delivery pump 700c as shown, which can be configured to deliver dextrose or a similar agent.

In some embodiments, delivery device 100a and 100b are configured in a client-server or other asymmetric communication arrangement (“client-server” herein), respectively, where delivery device 100a delivers an agent 200a comprising insulin, and delivery device 100b delivers an agent 200b comprising glucagon and/or glucose. Device 500 comprises a glucose monitoring device that provides (e.g. wirelessly provides) information (e.g. on a routine basis such as every 5 minutes) to delivery device 100a (e.g. to its algorithm 141) and/or another component of system 10. In an alternative embodiment, device 100a and/or 100b includes an integrated glucose sensing assembly (e.g. sensing assembly 170 described herein) that provides the glucose level data. Delivery device 100a, and/or another component of system 10, can provide dosing instructions (e.g. glucagon dosing instructions) to delivery device 100b. Fluid delivery pump 700c, can deliver dextrose to the patient, such as delivery also based on instructions received from delivery device 100a and/or another component of system 10. Communication device 300 can be configured as a control interface to delivery device 100 and/or other components of system 10, such as when communication device 300 is wirelessly connected to the associated component. Network 800 is configured for backing up data produced by the system 10 components, for data sharing, and/or data processing.

System 10 can be configured as a glycemic control system with counterregulatory control (provided by the glucagon delivery or, for patients in the ICU, a glucose infusion), such as when controlled by delivery device 100a.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for delivering an agent to a patient, comprising:
a delivery device configured to deliver a first agent to the patient, the delivery device comprising:
a reservoir for storing the first agent;
a transcutaneous delivery assembly for delivering the first agent to the patient transcutaneously;
a pumping mechanism for receiving the first agent from the reservoir and propelling the first agent to the transcutaneous delivery assembly;
a control module for controlling at least the pumping mechanism;
a power supply for providing energy to at least the control module and the pumping mechanism; and
a housing surrounding at least the pumping mechanism.

2.-64. (canceled)
